(19) United States
(12) Patent Application Publication
(10) Pub. No.: US 2023/0190151 A1
(43) Pub. Date: Jun. 22, 2023

(54) ANALYTE SENSOR DEPLOYMENT TESTING

(71) Applicant: Dexcom, Inc., San Diego, CA (US)

(72) Inventors: James S. Amidei, San Diego, CA (US); Stefan M. Robert, San Diego, CA (US); Anna Harley-Trochimczyk, San Diego, CA (US); Thomas George O'Connell, San Diego, CA (US); Anderson Ionut Micu, San Diego, CA (US)

(21) Appl. No.: 18/067,427

(22) Filed: Dec. 16, 2022

Related U.S. Application Data
(60) Provisional application No. 63/265,665, filed on Dec. 17, 2021.

(51) Int. Cl. A61B 5/145

(52) U.S. Cl. CPC A61B 5/14546; A61B 5/14532

(57) ABSTRACT

Various examples are directed to systems and methods that may utilize an analyte sensor system comprising a sensor enclosure; an analyte sensor extending from the sensor enclosure; and sensor electronics positioned within the sensor enclosure. The sensor electronics may be configured to detect that a wireless signal has changed from a first state to a second state, where the wireless signal may be provided through the sensor enclosure. After detecting that the wireless signal has changed from the first state to the second state, the sensor electronics may monitor whether the wireless signal remains in the second state for at least a stability threshold time period. The sensor electronics may execute a responsive action in the sensor system based at least in part on whether the wireless signal remains in the second state for at least the stability threshold time period.

### RELATED CASE(S)

This application claims the benefit of U.S. Provisional Application Ser. No. 63/265,665, filed on Dec. 17, 2021, which is incorporated herein by reference in its entirety.

### TECHNICAL FIELD

The present development relates generally to medical devices such as analyte sensors, and more particularly, but not by way of limitation, to systems, devices, and methods for testing analyte sensor deployment.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels. A glucose sensor can provide an estimated glucose concentration level, which can be used as guidance by a patient or caregiver.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Blood sugar concentration levels may be monitored with an analyte sensor, such as a continuous glucose monitor. A continuous glucose monitor is used by a host (e.g., patient) to provide information, such as an estimated blood glucose value or a trend of estimated blood glucose levels.

### SUMMARY

This present application discloses, among other things, systems, devices, and methods related to analyte sensor, including, for example, deploy testing in analyte sensors.

Example 1 is a sensor system for in vivo use, the sensor system comprising: a sensor enclosure; an analyte sensor extending from the sensor enclosure; and sensor electronics positioned within the sensor enclosure, the sensor electronics being configured to perform operations comprising: detecting that a wireless signal has changed from a first state to a second state, the wireless signal being provided through the sensor enclosure; after detecting that the wireless signal has changed from the first state to the second state, monitoring whether the wireless signal remains in the second state for at least a stability threshold time period; and executing an action in the sensor system, the action being selected based at least in part on whether the wireless signal remains in the second state for at least the stability threshold time period.

In Example 2, the subject matter of Example 1 optionally includes an applicator for inserting the analyte sensor under a skin of a host, wherein the applicator comprises a transmitter, and wherein the transmitter generates the wireless signal.

In Example 3, the subject matter of any one or more of Examples 1-2 optionally includes a wireless signal sensor, the wireless signal sensor comprising at least one of a magnetic signal sensor, an inductive signal sensor, or an optical signal sensor.

In Example 4, the subject matter of any one or more of Examples 1-3 optionally includes the detecting that the wireless signal has changed from the first state to the second state comprising: determining, at a first time that the wireless signal is in the first state at least in part by determining that the wireless signal is present; and determining, at a second time, that the wireless signal is in the second state at least in part by determining that the wireless signal is absent, the second time being after the first time.

In Example 5, the subject matter of any one or more of Examples 1˜4 optionally includes the detecting that the wireless signal has changed from the first state to the second state comprising: determining, at a first time, that the wireless signal is in the first state at least in part by determining that the wireless signal has a first magnetic polarity; and determining, at a second time, that the wireless signal is in the second state at least in part by determining that the wireless signal has a second magnetic polarity different than the first magnetic polarity, the second time being after the first time.

In Example 6, the subject matter of any one or more of Examples 1-5 optionally includes wherein determining whether the wireless signal remains in the second state for at least the stability threshold time period comprises determining that the wireless signal does remain in the second state for at least the stability threshold time period, and wherein the action comprises determining an estimated analyte value for a host using a sensor output generated by the analyte sensor.

In Example 7, the subject matter of any one or more of Examples 1-6 optionally includes the operations further comprising determining that the sensor electronics have established a communication connection with an external device before the wireless signal has remained in the second state for at least the stability threshold time period, wherein the action comprises determining an estimated analyte value for a host using a sensor output generated by the analyte sensor.

In Example 8, the subject matter of any one or more of Examples 1-7 optionally includes the operations further comprising after detecting that the wireless signal has changed from the first state to the second state, determining that the wireless signal has remained in the first state for more than the stability threshold time period, the action comprising configuring the sensor system to a sleep mode.

In Example 9, the subject matter of any one or more of Examples 1-8 optionally includes the operations further comprising determining that a startup time period has passed since the detecting that the wireless signal has changed from the first state to the second state.

In Example 10, the subject matter of any one or more of Examples 1-9 optionally includes the operations further comprising determining that more than a failure threshold time period has passed since the detecting that the wireless signal has changed from the first state to the second state, the failure threshold time period being greater than the stability threshold time period.

In Example 11, the subject matter of any one or more of Examples 1-10 optionally includes the operations further comprising: determining that a startup threshold time period has passed since the detecting that the wireless signal has changed from the first state to the second state; and determining that the wireless signal has remained in the first state for more than the stability threshold time period, the action comprising configuring the sensor system to a failure state.

In Example 12, the subject matter of any one or more of Examples 1-11 optionally includes the operations further comprising: determining that a startup threshold time period has passed since the detecting that the wireless signal has changed from the first state to the second state and that the wireless signal has not remained in either the first state or in the second state for more than the stability threshold time period; and initiating a communication connection with an external device.

In Example 13, the subject matter of Example 12 optionally includes the operations further comprising determining that the sensor electronics have established the communication connection with the external device, wherein the action comprises determining an estimated analyte value for a host using a sensor output generated by the analyte sensor.

In Example 14, the subject matter of any one or more of Examples 1-13 optionally includes the operations further comprising determining that a sensor output generated by the analyte sensor meets a first insertion condition.

In Example 15, the subject matter of Example 14 optionally includes the operations further comprising: after determining that the sensor output meets the first insertion condition, initiating a communication connection with an external device; and determining that the sensor electronics have established the communication connection with the external device, wherein the action comprises determining an estimated analyte value for a host using a sensor output generated by the analyte sensor.

Example 16 is a method for operating an analyte sensor system comprising an in vivo analyte sensor, the method comprising: detecting, by sensor electronics of the analyte sensor system, that a wireless signal has changed from a first state to a second state, the wireless signal being provided to the sensor electronics through a sensor enclosure of the analyte sensor system; after detecting that the wireless signal has changed from the first state to the second state, monitoring, by the sensor electronics, whether the wireless signal remains in the second state for at least a stability threshold time period; and executing, by the sensor electronics, an action in the sensor system, the action being selected based at least in part on whether the wireless signal remains in the second state for at least the stability threshold time period.

In Example 17, the subject matter of Example 16 optionally includes receiving the wireless signal, by the sensor electronics and from a transmitter, the transmitter positioned at an applicator for inserting an analyte sensor of the analyte sensor system under a skin of a host.

In Example 18, the subject matter of any one or more of Examples 16-17 optionally includes receiving the wireless signal using at least one of a magnetic signal sensor, an inductive signal sensor, or an optical signal sensor.

In Example 19, the subject matter of any one or more of Examples 16-18 optionally includes the detecting that the wireless signal has changed from the first state to the second state comprising: determining, at a first time that the wireless signal is in the first state at least in part by determining that the wireless signal is present; and determining, at a second time, that the wireless signal is in the second state at least in part by determining that the wireless signal is absent, the second time being after the first time.

In Example 20, the subject matter of any one or more of Examples 16-19 optionally includes the detecting that the wireless signal has changed from the first state to the second state comprising: determining, at a first time, that the wireless signal is in the first state at least in part by determining that the wireless signal has a first magnetic polarity; and determining, at a second time, that the wireless signal is in the second state at least in part by determining that the wireless signal has a second magnetic polarity different than the first magnetic polarity, the second time being after the first time.

In Example 21, the subject matter of any one or more of Examples 16-20 optionally includes wherein determining whether the wireless signal remains in the second state for at least the stability threshold time period comprises determining that the wireless signal does remain in the second state for at least the stability threshold time period, and wherein the action comprises determining an estimated analyte value for a host using a sensor output generated by the analyte sensor.

In Example 22, the subject matter of any one or more of Examples 16-21 optionally includes determining that the sensor electronics have established a communication connection with an external device before the wireless signal has remained in the second state for at least the stability threshold time period, wherein the action comprises determining an estimated analyte value for a host using a sensor output generated by the analyte sensor.

In Example 23, the subject matter of any one or more of Examples 16-22 optionally includes after detecting that the wireless signal has changed from the first state to the second state, determining that the wireless signal has remained in the first state for more than the stability threshold time period, the action comprising configuring the sensor system to a sleep mode.

In Example 24, the subject matter of any one or more of Examples 16-23 optionally includes determining that a startup time period has passed since the detecting that the wireless signal has changed from the first state to the second state.

In Example 25, the subject matter of any one or more of Examples 16-24 optionally includes determining that more than a failure threshold time period has passed since the detecting that the wireless signal has changed from the first state to the second state, the failure threshold time period being greater than the stability threshold time period.

In Example 26, the subject matter of any one or more of Examples 16-25 optionally includes determining that a startup threshold time period has passed since the detecting that the wireless signal has changed from the first state to the second state; and determining that the wireless signal has remained in the first state for more than the stability threshold time period, the action comprising configuring the sensor system to a failure state.

In Example 27, the subject matter of any one or more of Examples 16-26 optionally includes determining that a startup threshold time period has passed since the detecting that the wireless signal has changed from the first state to the second state and that the wireless signal has not remained in either the first state or in the second state for more than the stability threshold time period; and initiating a communication connection with an external device.

In Example 28, the subject matter of Example 27 optionally includes determining that the sensor electronics have established the communication connection with the external device, wherein the action comprises determining an estimated analyte value for a host using a sensor output generated by the analyte sensor.

In Example 29, the subject matter of any one or more of Examples 16-28 optionally includes determining that a sensor output generated by the analyte sensor meets a first insertion condition.

In Example 30, the subject matter of Example 29 optionally includes after determining that the sensor output meets the first insertion condition, initiating a communication connection with an external device; and determining that the sensor electronics have established the communication connection with the external device, wherein the action comprises determining an estimated analyte value for a host using a sensor output generated by the analyte sensor.

Example 31 is a non-transitory machine readable medium comprising instructions thereon that, when executed by an analyte sensor system, cause the analyte sensor system to perform operations comprising: detecting that a wireless signal has changed from a first state to a second state, the wireless signal being provided to sensor electronics of the analyte sensor system through a sensor enclosure of the analyte sensor system; after detecting that the wireless signal has changed from the first state to the second state, monitoring whether the wireless signal remains in the second state for at least a stability threshold time period; and executing an action in the sensor system, the action being selected based at least in part on whether the wireless signal remains in the second state for at least the stability threshold time period.

In Example 32, the subject matter of Example 31 optionally includes the operations further comprising receiving the wireless signal from a transmitter, the transmitter positioned at an applicator for inserting an analyte sensor of the analyte sensor system under a skin of a host.

In Example 33, the subject matter of any one or more of Examples 31-32 optionally includes the operations further comprising receiving the wireless signal using at least one of a magnetic signal sensor, an inductive signal sensor, or an optical signal sensor.

In Example 34, the subject matter of any one or more of Examples 31-33 optionally includes the detecting that the wireless signal has changed from the first state to the second state comprising: determining, at a first time that the wireless signal is in the first state at least in part by determining that the wireless signal is present; and determining, at a second time, that the wireless signal is in the second state at least in part by determining that the wireless signal is absent, the second time being after the first time.

In Example 35, the subject matter of any one or more of Examples 31-34 optionally includes the detecting that the wireless signal has changed from the first state to the second state comprising: determining, at a first time, that the wireless signal is in the first state at least in part by determining that the wireless signal has a first magnetic polarity; and determining, at a second time, that the wireless signal is in the second state at least in part by determining that the wireless signal has a second magnetic polarity different than the first magnetic polarity, the second time being after the first time.

In Example 36, the subject matter of any one or more of Examples 31-35 optionally includes wherein determining whether the wireless signal remains in the second state for at least the stability threshold time period comprises determining that the wireless signal does remain in the second state for at least the stability threshold time period, and wherein the action comprises determining an estimated analyte value for a host using a sensor output generated by the analyte sensor.

In Example 37, the subject matter of any one or more of Examples 31-36 optionally includes the operations further comprising determining that the sensor electronics have established a communication connection with an external device before the wireless signal has remained in the second state for at least the stability threshold time period, wherein the action comprises determining an estimated analyte value for a host using a sensor output generated by the analyte sensor.

In Example 38, the subject matter of any one or more of Examples 31-37 optionally includes the operations further comprising, after detecting that the wireless signal has changed from the first state to the second state, determining that the wireless signal has remained in the first state for more than the stability threshold time period, the action comprising configuring the sensor system to a sleep mode.

In Example 39, the subject matter of any one or more of Examples 31-38 optionally includes the operations further comprising determining that a startup time period has passed since the detecting that the wireless signal has changed from the first state to the second state.

In Example 40, the subject matter of any one or more of Examples 31-39 optionally includes the operations further comprising determining that more than a failure threshold time period has passed since the detecting that the wireless signal has changed from the first state to the second state, the failure threshold time period being greater than the stability threshold time period.

In Example 41, the subject matter of any one or more of Examples 31-40 optionally includes the operations further comprising: determining that a startup threshold time period has passed since the detecting that the wireless signal has changed from the first state to the second state; and determining that the wireless signal has remained in the first state for more than the stability threshold time period, the action comprising configuring the sensor system to a failure state.

In Example 42, the subject matter of any one or more of Examples 31-41 optionally includes the operations further comprising: determining that a startup threshold time period has passed since the detecting that the wireless signal has changed from the first state to the second state and that the wireless signal has not remained in either the first state or in the second state for more than the stability threshold time period; and initiating a communication connection with an external device.

In Example 43, the subject matter of Example 42 optionally includes the operations further comprising determining that the sensor electronics have established the communication connection with the external device, wherein the action comprises determining an estimated analyte value for a host using a sensor output generated by the analyte sensor.

In Example 44, the subject matter of any one or more of Examples 31-43 optionally includes the operations further comprising determining that a sensor output generated by the analyte sensor meets a first insertion condition.

In Example 45, the subject matter of Example 44 optionally includes the operations further comprising: after determining that the sensor output meets the first insertion condition, initiating a communication connection with an external device; and determining that the sensor electronics have established the communication connection with the external device, wherein the action comprises determining an estimated analyte value for a host using a sensor output generated by the analyte sensor.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments described in the present document.

FIG. 1 is a diagram showing one example of an environment including an analyte sensor system.

FIG. 2 is a diagram showing one example of a medical device system including the analyte sensor system of FIG. 1.

FIG. 3 is an illustration of an example analyte sensor.

FIG. 4 is an illustration of another example analyte sensor.

FIG. 5 is an enlarged view of an example analyte sensor portion.

FIG. 6 is a cross-sectional view of the analyte sensor of FIGS. 3 and 4.

FIG. 7 is a schematic illustration of a circuit that represents the behavior of an example analyte sensor.

FIG. 8 is an exploded side view showing one example of a sensor applicator.

FIG. 9A shows one example of the needle and sensor of FIG. 8 loaded prior to sensor insertion.

FIG. 9B shows one example of the needle and sensor after sensor insertion.

FIG. 9C shows one example of the sensor and needle during needle retraction.

FIG. 9D shows one example of the sensor remaining within the contact subassembly after needle retraction.

FIG. 10 is a perspective view of the sensor applicator of FIG. 9 and a mounting unit according to one example including a safety latch mechanism.

FIG. 11 is a diagram showing another example of an analyte sensor applicator.

FIG. 12 is a diagram showing one example of an analyte sensor system including an analyte sensor and sensor electronics.

FIG. 13 is a flowchart showing one example of a process flow that may be executed by the sensor electronics of the sensor system of FIG. 12 to transition the sensor system from a sleep mode to an active mode.

FIG. 14 is a state diagram showing one example of a workflow that may be executed by the sensor electronics of the sensor system of FIG. 12 to perform an insertion test.

FIG. 15 is a state diagram showing one example of a workflow that may be executed by the sensor electronics of the sensor system of FIG. 12 to perform a deploy test to test for deployment of the analyte sensor using the wireless signal detected by the deployment sensor.

FIG. 16 is a state diagram showing an example workflow that may be executed by the sensor electronics to transition the sensor system from a sleep mode to an active mode.

FIG. 17 is state diagram showing another example workflow that may be executed by the sensor electronics to transition the sensor system from a sleep mode to an active mode.

FIG. 18 is a block diagram illustrating a computing device hardware architecture, within which a set or sequence of instructions can be executed to cause a machine to perform examples of any one of the methodologies discussed herein.

### DETAILED DESCRIPTION

Various examples described herein are directed to analyte sensor systems and methods for using analyte sensor systems. An analyte sensor system includes an analyte sensor that is placed in contact with a bodily fluid of a host to measure a concentration of an analyte, such as glucose, in the bodily fluid. In some examples, the analyte sensor is inserted into the host to contact the bodily fluid in vivo. In some examples, the analyte sensor is inserted subcutaneously to contact interstitial fluid below the host's skin.

When the analyte sensor is exposed to analyte in the host's bodily fluid, an electrochemical reaction between the analyte sensor and the analyte causes the analyte sensor to generate a raw sensor signal that is indicative of the analyte concentration in the bodily fluid. For example, the analyte sensor may include two or more electrodes. An analyte sensor system control circuit applies a bias condition to the electrodes. The bias condition may be, for example, a potential difference applied between a working electrode of the analyte sensor and a reference electrode of the analyte sensor. The bias condition promotes the electrochemical reaction between the analyte and the analyte sensor, resulting in a current between the working electrode and at least one other analyte sensor electrode. The raw sensor signal may be and/or may be based on the current.

The control circuit uses the raw sensor signal to determine an estimated analyte value. In some examples, the control circuit is also programmed to output result data, which may include the estimated analyte value or other data. In some examples, the control circuit communicates result data to one or more other external devices.

After manufacturing, an analyte sensor system may be transported and stored prior to use by a host. During transport and storage, the analyte sensor system may be configured to operate in a sleep mode. In the sleep mode, the analyte sensor system is configured to minimize power usage, thereby saving battery or other onboard power for when the sensor system is in active use by a host. When a host is ready to use the analyte sensor, the host inserts the sensor in vivo, for example, as described herein. After the analyte sensor is positioned in vivo, the control circuit may begin to operate the sensor in an active or measuring state. In the active or measuring state, the control circuit may apply the bias condition to the analyte sensor and/or determine an estimated analyte value values using the raw sensor signal generated by the sensor. In some examples, the control circuit may also initiate communication connections with one or more other external devices to provide the one or more other external devices with result data.

The control circuit, then, may be configured to detect when the analyte sensor is inserted in vivo to transition the analyte sensor system from the sleep mode to the active mode. One example way to detect sensor insertion is to periodically monitor the sensor to detect the presence of a raw sensor signal, e.g., a signal determined to have one or more characteristics, conditions, and/or profiles. For example, the analyte sensor may generate some level of raw sensor signal upon insertion. The control circuit may detect insertion by determining that the analyte sensor is generating a raw sensor signal that meets an insertion condition such as, for example, current greater than a threshold value for greater than a threshold time. In some examples, however, the analyte sensor may not begin generating a raw sensor signal sufficient to indicate in vivo insertion until sometime after insertion has taken place, in some examples in excess of two minutes or more. As a result, the control circuit may not operate the analyte sensor system in the active mode until portions of an initial sensor deployment period have passed. Also, in some examples, the raw sensor signal generated before the control circuit applies a bias condition may not be sufficient to accurately detect insertion.

In some examples, the control circuit is configured to detect insertion using a deployment sensor. A deployment sensor may generate a signal indicating when the sensor is deployed. Using a deployment sensor, however, may pose additional challenges. For example, the control circuit of the analyte sensor may be enclosed within a sensor enclosure, with the analyte sensor extending from the sensor enclosure. When the analyte sensor is inserted in vivo, the sensor enclosure may be positioned on and adhered to the surface of the host's skin, as described herein. Accordingly, the sensor enclosure may be exposed to bodily fluids from the skin below and/or to material and fluids that may otherwise come into contact with the host's skin.

For at least these reasons, it may be desirable to seal the sensor enclosure. Sealing the sensor enclosure, however, may pose challenges when using a mechanical deployment sensor, such as a switch. For example, a mechanical deployment sensor may extend through the enclosure, preventing and/or complicating the sealing of the enclosure. A mechanical deployment sensor may also generate bounce artifacts in the raw sensor signal that can waste system power and extend the time to detect insertion.

Some examples described herein use a deployment sensor that receives a wireless signal that is transmitted through the sensor enclosure. The wireless signal may have a first state indicating that the analyte sensor is undeployed. The first state may be referred to as an undeployed state. The wireless signal may also have a second state indicating that the analyte sensor is deployed. The second state may be referred to as a deployed state.

The wireless signal may be generated and/or transmitted by a transmitter. For example, the transmitter may be positioned at a sensor applicator for inserting the analyte sensor. Prior to insertion of the analyte sensor, the transmitter may provide the wireless signal in the undeployed state. After the analyte sensor is deployed in vivo, the transmitter may change the wireless signal to the deployed state.

The sensor control circuit may comprise a deployment sensor that is configured to receive the wireless signal. When the analyte sensor system is in the sleep mode, the sensor control circuit may periodically poll the deployment sensor to determine the state of the wireless signal. When the wireless signal changes from the undeployed state to the deployed state, the sensor control circuit may begin operating the sensor system in the active mode.

Various types of wireless technologies may be used to implement the transmitter and the deployment sensor. In some examples, the transmitter is or includes a permanent magnet, solenoid, or other device for generating a magnetic field. The receiver may be or comprise a solenoid, inductor, or other device that can detect a magnetic field. The magnetic field may make up all or part of the wireless signal. For example, the first state of the wireless signal may occur when the magnetic field has a first polarity, and the second state of the wireless signal may occur when the magnetic field has a second polarity different than the first polarity. In another example, the first state of the wireless signal may be indicated by the presence of the magnetic field while the second state is indicated by the absence (or relative absence) of the magnetic field. In another example, the first state is indicated by the absence (or relative absence) of the magnetic field and the second state is indicated by the presence of other magnetic field. Other wireless technologies that may be used to generate the wireless signal include, for example, optical transmitters and receivers, Radio Frequency Identification (RFID) transmitters and receivers, and the like.

Using a wireless signal transmitter, however, may generate other challenges. For example, when the transmitter is included in an analyte sensor applicator, the transition of the transmitter between transmitting the undeployed state of the wireless signal and the deployed state of the wireless signal may be influenced by the actions of the host or other user. For example, the host or other user may not always cleanly remove the applicator upon insertion of the analyte sensor. For example, the host or other user may wiggle the applicator to disconnect it from the sensor enclosure. Also, sometimes, the host or other user may use the applicator to help secure the sensor enclosure to the host's skin. In some examples, these and similar actions may cause the transmitter to toggle the wireless signal between the deployed and undeployed states and/or may cause the transmitter to settle the wireless signal into the undeployed state even if the analyte sensor is actually inserted under the host's skin. Also, in some examples, the wireless signal is influenced by other environmental signals, such as light in the environment, devices that generate electromagnetic fields, and the like. This may cause the wireless signal to toggle to a state that is inconsistent with the actual condition of the analyte sensor.

In some examples, the control circuit is configured to test the wireless signal to determine when the analyte sensor has been inserted and the sensor system should be operated in the active mode.

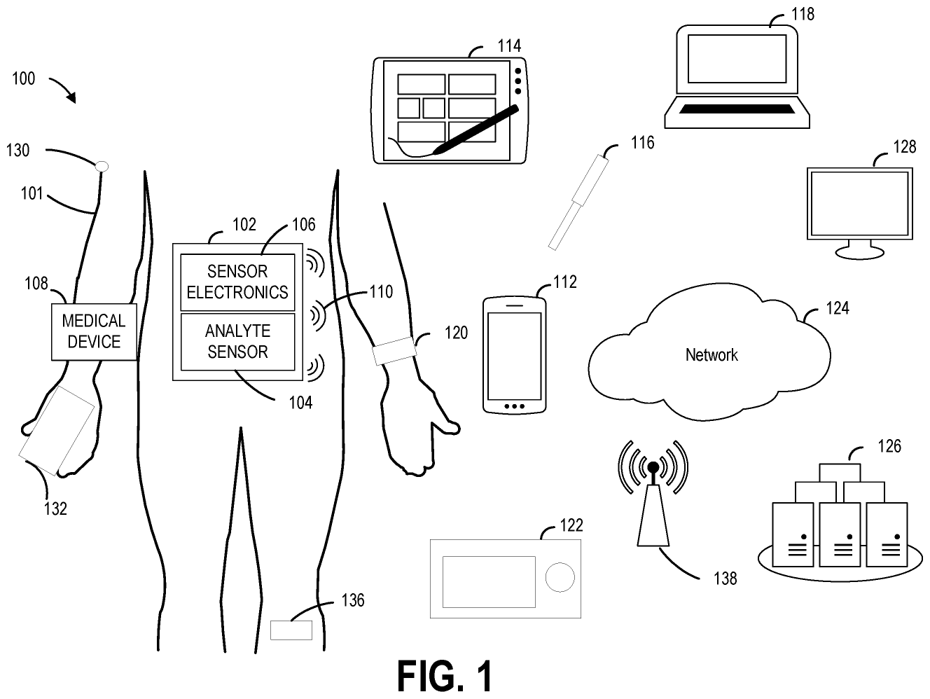

FIG. 1 is a diagram showing one example of an environment 100 including an analyte sensor system 102. The analyte sensor system 102 is coupled to a host 101, which may be a human patient. In some examples, the host is subject to a temporary or permanent diabetes condition or other health condition that makes analyte monitoring useful.

The analyte sensor system 102 includes an analyte sensor 104. In some examples, the analyte sensor 104 is or includes a glucose sensor configured to measure a glucose concentration in the host 101. The analyte sensor 104 can be exposed to analyte at the host 101 in any suitable way. In some examples, the analyte sensor 104 is fully implantable under the skin of the host 101. In other examples, the analyte sensor 104 is wearable on the body of the host 101 (e.g., on the body but not under the skin). Also, in some examples, the analyte sensor 104 is a transcutaneous device (e.g., with a sensor residing at least partially under or in the skin of a host). It should be understood that the devices and methods described herein can be applied to any device capable of detecting a concentration of an analyte, such as glucose, and providing an output signal that represents the concentration of the analyte.

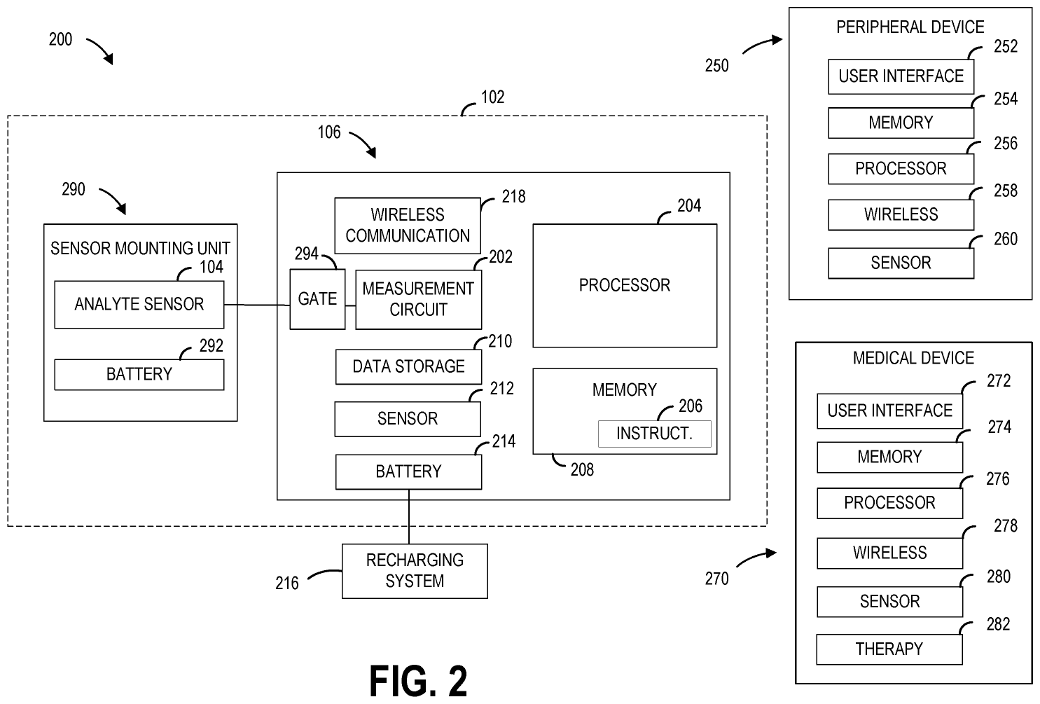

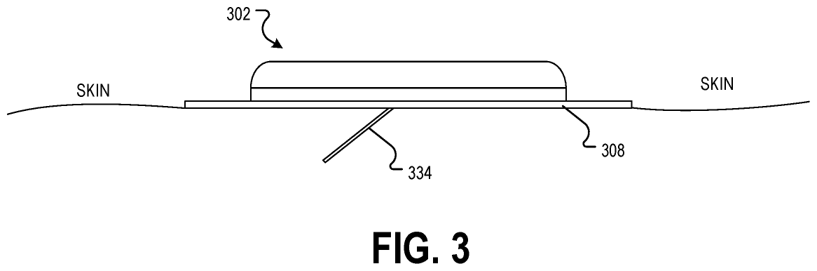

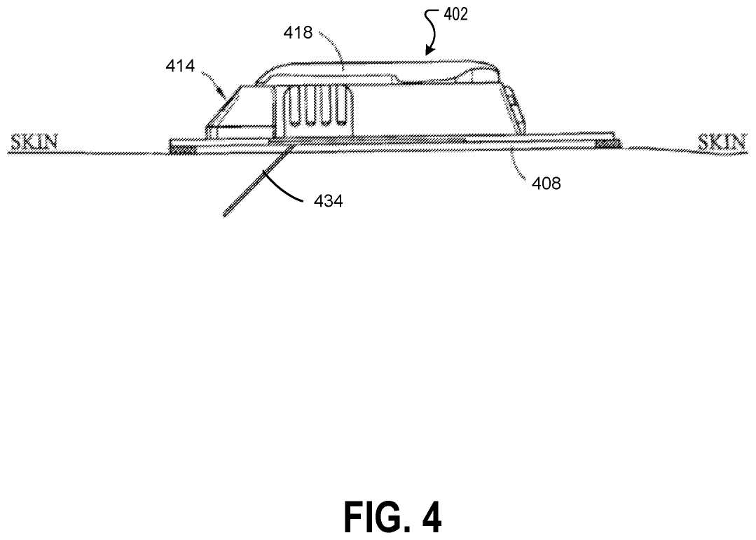

In the example of FIG. 1, the analyte sensor system 102 also includes sensor electronics 106. In some examples, the sensor electronics 106 and analyte sensor 104 are provided in a single integrated enclosure (See FIG. 3). In other examples, the analyte sensor 104 and sensor electronics 106 are provided as separate components or modules (See FIG. 4). For example, the analyte sensor system 102 may include a disposable (e.g., single-use) sensor mounting unit that may include the analyte sensor 104, a component for attaching the sensor 104 to a host (e.g., an adhesive pad), and/or a mounting structure configured to receive a sensor electronics unit including some or all of the sensor electronics 106 shown in FIG. 2. The sensor electronics unit may be reusable.

The analyte sensor 104 may use any known method, including invasive, minimally-invasive, or non-invasive sensing techniques (e.g., optically excited fluorescence, microneedle, transdermal monitoring of glucose), to provide a raw sensor signal indicative of the concentration of the analyte in the host 101. The raw sensor signal may be converted into calibrated and/or filtered analyte concentration data used to provide a useful value of the analyte concentration (e.g., estimated blood glucose concentration level) to a user, such as the host or a caretaker (e.g., a parent, a relative, a guardian, a teacher, a doctor, a nurse, or any other individual that has an interest in the wellbeing of the host 101).

In some examples, the analyte sensor 104 is or includes a continuous glucose sensor. A continuous glucose sensor can be or include a subcutaneous, transdermal (e.g., transcutaneous), and/or intravascular device. In some embodiments, such a sensor or device may recurrently (e.g., periodically, or intermittently) analyze sensor data. The glucose sensor may use any method of glucose measurement, including enzymatic, chemical, physical, electrochemical, spectrophotometric, polarimetric, calorimetric, iontophoretic, radiometric, immunochemical, and the like. In various examples, the analyte sensor system 102 may be or include a continuous glucose monitor sensor available from DexCom™, (e.g., the DexCom G5™ sensor, Dexcom G6™ sensor, the DexCom G7™ sensor, or any variation thereof), from Abbott™ (e.g., the Libre™ sensor), or from Medtronic™ (e.g., the Enlite™ sensor).

In some examples, analyte sensor 104 includes an implantable glucose sensor, such as described with reference to U.S. Pat. No. 6,001,067 and U.S. Patent Publication No. US-2005-0027463-A1, which are incorporated by reference. In some examples, analyte sensor 104 includes a transcutaneous glucose sensor, such as described with reference to U.S. Patent Publication No. US-2006-0020187-A1, which is incorporated by reference. In some examples, analyte sensor 104 may be configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007, all of which are incorporated by reference. In some examples, the continuous glucose sensor may include a transcutaneous sensor such as described in U.S. Pat. No. 6,565,509 to Say et al., which is incorporated by reference. In some examples, analyte sensor 104 may include a continuous glucose sensor that includes a subcutaneous sensor such as described with reference to U.S. Pat. No. 6,579,690 to Bonnecaze et al. or U.S. Pat. No. 6,484,046 to Say et al., which are incorporated by reference. In some examples, the continuous glucose sensor may include a refillable subcutaneous sensor such as described with reference to U.S. Pat. No. 6,512,939 to Colvin et al., which is incorporated by reference. The continuous glucose sensor may include an intravascular sensor such as described with reference to U.S. Pat. No. 6,477,395 to Schulman et al., which is incorporated by reference. The continuous glucose sensor may include an intravascular sensor such as described with reference to U.S. Pat. No. 6,424,847 to Mastrototaro et al., which is incorporated by reference.

The environment 100 may also include various other external devices including, for example, a medical device 108. The medical device 108 may be or include a drug delivery device such as an insulin pump or an insulin pen. In some examples, the medical device 108 includes one or more sensors, such as another analyte sensor, a heart rate sensor, a respiration sensor, a motion sensor (e.g., accelerometer), posture sensor (e.g., 3-axis accelerometer), acoustic sensor (e.g., to capture ambient sound or sounds inside the body). The medical device 108 may be wearable, e.g., on a watch, glasses, contact lens, patch, wristband, ankle band, or another wearable item, or may be incorporated into a handheld device (e.g., a smartphone). In some examples, the medical device 108 includes a multi-sensor patch that may, for example, detect one or more of an analyte levels (e.g., glucose, lactate, insulin, or other substance), heart rate, respiration (e.g., using impedance), activity (e.g., using an accelerometer), posture (e.g., using an accelerometer), galvanic skin response, tissue fluid levels (e.g., using impedance or pressure).

In some examples, the analyte sensor system 102 and the medical device 108 communicate with one another. Communication between the analyte sensor system 102 and medical device 108 may occur over any suitable wired connection and/or via a wireless communication signal 110. For example, the analyte sensor system 102 (e.g., the sensor electronics 106 thereof) may be configured to establish a communication connection with the medical device 108 using a suitable short-range communications medium such as, for example, a radio frequency medium (e.g., Bluetooth, Medical Implant Communication System (MICS), Wi-Fi, near field communication (NFC), radio frequency identification (RFID), Zigbee, Z-Wave or other communication protocols), an optical medium (e.g., infrared), a sonic medium (e.g., ultrasonic), a cellular protocol-based medium (e.g., Code Division Multiple Access (CDMA) or Global System for Mobiles (GSM)), and/or the like.

In some examples, the environment 100 also includes other external devices such as, for example, a wearable sensor 130. The wearable sensor 130 can include a sensor circuit (e.g., a sensor circuit configured to detect a glucose concentration or other analyte concentration) and a communication circuit, which may, for example, be an NFC circuit. In some examples, information from the wearable sensor 130 may be retrieved from the wearable sensor 130 using a user computing device 132, such as a smart phone, that is configured to communicate with the wearable sensor 130 via the wearable sensor's communication circuit, for example, when the user device 132 is placed near the wearable sensor 130. For example, swiping the user device 132 over the sensor 130 may retrieve sensor data from the wearable sensor 130 using NFC or other suitable wireless communication.

The use of NFC communication may reduce power consumption by the wearable sensor 130, which may reduce the size of a power source (e.g., battery or capacitor) in the wearable sensor 130 or extend the usable life of the power source. In some examples, the wearable sensor 130 may be wearable on an upper arm as shown. In some examples, a wearable sensor 130 may additionally or alternatively be on the upper torso of the patient (e.g., over the heart or over a lung), which may, for example, facilitate detecting heart rate, respiration, or posture. A wearable sensor 136 may also be on the lower body (e.g., on a leg) or other part of the body (e.g., on the abdomen).

In some examples, an array or network of sensors may be associated with the patient. For example, one or more of the analyte sensor system 102, and/or external devices, such as the medical device 108, wearable device 120 such as a watch, an additional wearable sensor 130 and/or the like, may communicate with one another via a short-range communication medium (e.g., Bluetooth, MICS, NFC, or any of the other options described above). The additional wearable sensor 130 may be any of the examples described above with respect to medical device 108. The analyte sensor system 102, medical device 108, and additional sensor 130 on the host 101 are provided for illustration and description and are not necessarily drawn to scale.

The environment 100 may also include one or more other external devices such as a hand-held smart device (e.g., smart phone) 112, tablet 114, smart pen 116 (e.g., insulin delivery pen with processing and communication capability), computer 118, a wearable device 120 such as a watch, or peripheral medical device 122 (which may be a proprietary device such as a proprietary user device available from DexCom™), any of which may communicate with the analyte sensor system 102 via a short-range communication medium, such as indicated by wireless communication signal 110, and may also communicate over a network 124 with a server system (e.g., remote data center) or with a remote terminal 128 to facilitate communication with a remote user (not shown) such as a technical support staff member or a clinician.

The wearable device 120 may include an activity sensor, a heart rate monitor (e.g., light-based sensor or electrode-based sensor), a respiration sensor (e.g., acoustic- or electrode-based), a location sensor (e.g., GPS), or other sensors.

In some examples, the environment 100 includes a server system 126. The server system 126 can include one or more computing devices, such as one or more server computing devices. In some examples, the server system 126 is used to collect analyte data from the analyte sensor system 102 and/or analyte or other data from the plurality of other devices, and to perform analytics on collected data, generate, or apply universal or individualized models for glucose levels, and communicate such analytics, models, or information based thereon back to one or more of the devices in the environment 100. In some examples, the server system 126 gathers inter-host and/or intra-host break-in data to generate one or more break-in characteristics, as described herein.

The environment 100 may also include a wireless access point (WAP) 138 used to communicatively couple one or more of analyte sensor system 102, network 124, server system 126, medical device 108 or any of the peripheral devices described above. For example, WAP 138 may provide Wi-Fi and/or cellular connectivity within environment 100. Other communication protocols, such as NFC or Bluetooth, may also be used among devices of the environment 100.

FIG. 2 is a diagram showing one example of a medical device system 200 including the analyte sensor system 102 of FIG. 1. In the example of FIG. 2, the analyte sensor system 102 includes sensor electronics 106 and an example sensor mounting unit 290, although in some examples, it will be appreciated that the analyte sensor 104 and sensor electronics 106 may be included in a common enclosure. While a specific example of division of components between the sensor mounting unit 290 and sensor electronics 106 is shown, it is understood that some examples may include additional components in the sensor mounting unit 290 or in the sensor electronics 106, and that some of the components (e.g., a battery or supercapacitor) that are shown in the sensor electronics 106 may be alternatively or additionally (e.g., redundantly) provided in the sensor mounting unit 290.

In the example shown in FIG. 2, the sensor mounting unit 290 includes the analyte sensor 104 and a battery 292. In some examples, the sensor mounting unit 290 may be replaceable, and the sensor electronics 106 may include a debouncing circuit (e.g., gate with hysteresis or delay) to avoid, for example, recurrent execution of a power-up or power down process when a battery is repeatedly connected and disconnected or avoid processing of noise signal associated with removal or replacement of a battery.

The sensor electronics 106 may include electronics components that are configured to process sensor information, such as raw sensor signals, and generate corresponding analyte concentration values. The sensor electronics 106 may, for example, include electronic circuitry associated with measuring, processing, storing, or communicating continuous analyte sensor data, including prospective algorithms associated with processing and calibration of the raw sensor signal. The sensor electronics 106 may include hardware, firmware, and/or software that enables measurement of levels of the analyte via a glucose sensor. Electronic components may be affixed to a printed circuit board (PCB), or the like, and can take a variety of forms. For example, the electronic components may take the form of an integrated circuit (IC), such as an Application-Specific Integrated Circuit (ASIC), a microcontroller, and/or a processor.

In the example of FIG. 2, the sensor electronics 106 include a measurement circuit 202 (e.g., potentiostat) coupled to the analyte sensor 104 and configured to recurrently obtain analyte sensor readings using the analyte sensor 104. For example, the measurement circuit 202 may continuously or recurrently measure a raw sensor signal indicating a current flow at the analyte sensor 104 between a working electrode and a reference electrode. The sensor electronics 106 may include a gate circuit 294, which may be used to gate the connection between the measurement circuit 202 and the analyte sensor 104. For example, the analyte sensor 104 may accumulate charge over an accumulation period. After the accumulation period, the gate circuit 294 is opened so that the measurement circuit 202 can measure the accumulated charge. Gating the analyte sensor 104 may improve the performance of the sensor system 102 by creating a larger signal to noise or interference ratio (e.g., because charge accumulates from an analyte reaction, but sources of interference, such as the presence of acetaminophen near a glucose sensor, do not accumulate, or accumulate less than the charge from the analyte reaction).

The sensor electronics 106 may also include a processor 204. The processor 204 is configured to retrieve instructions 206 from memory 208 and execute the instructions 206 to control various operations in the analyte sensor system 102. For example, the processor 204 may be programmed to control application of bias potentials to the analyte sensor 104 via a potentiostat at the measurement circuit 202, interpret raw sensor signals from the analyte sensor 104, and/or compensate for environmental factors.

The processor 204 may also save information in data storage memory 210 or retrieve information from data storage memory 210. In various examples, data storage memory 210 may be integrated with memory 208, or may be a separate memory circuit, such as a non-volatile memory circuit (e.g., flash RAM). Examples of systems and methods for processing sensor analyte data are described in more detail herein and in U.S. Pat. Nos. 7,310,544 and 6,931,327.

The sensor electronics 106 may also include one or more sensors, such as the sensor 212, which may be coupled to the processor 204. The sensor 212 may be a temperature sensor, accelerometer, or another suitable sensor. The sensor electronics 106 may also include a power source such as a capacitor or battery 214, which may be integrated into the sensor electronics 106, or may be removable, or part of a separate electronics unit. The battery 214 (or other power storage component, e.g., capacitor) may optionally be rechargeable via a wired or wireless (e.g., inductive or ultrasound) recharging system 216. The recharging system 216 may harvest energy or may receive energy from an external source or on-board source. In various examples, the recharge circuit may include a triboelectric charging circuit, a piezoelectric charging circuit, an RF charging circuit, a light charging circuit, an ultrasonic charging circuit, a heat charging circuit, a heat harvesting circuit, or a circuit that harvests energy from the communication circuit. In some examples, the recharging circuit may recharge the rechargeable battery using power supplied from a replaceable battery (e.g., a battery supplied with a base component).

The sensor electronics 106 may also include one or more supercapacitors in the sensor electronics unit (as shown), or in the sensor mounting unit 290. For example, the supercapacitor may allow energy to be drawn from the battery 214 in a highly consistent manner to extend the life of the battery 214. The battery 214 may recharge the supercapacitor after the supercapacitor delivers energy to the communication circuit or to the processor 204, so that the supercapacitor is prepared for delivery of energy during a subsequent high-load period. In some examples, the supercapacitor may be configured in parallel with the battery 214. A device may be configured to preferentially draw energy from the supercapacitor, as opposed to the battery 214. In some examples, a supercapacitor may be configured to receive energy from a rechargeable battery for short-term storage and transfer energy to the rechargeable battery for long-term storage.

The supercapacitor may extend an operational life of the battery 214 by reducing the strain on the battery 214 during the high-load period. In some examples, a supercapacitor removes at least 10% of the strain off the battery during high-load events. In some examples, a supercapacitor removes at least 20% of the strain off the battery during high-load events. In some examples, a supercapacitor removes at least 30% of the strain off the battery during high-load events. In some examples, a supercapacitor removes at least 50% of the strain off the battery during high-load events.

The sensor electronics 106 may also include a wireless communication circuit 218, which may for example include a wireless transceiver operatively coupled to an antenna. The wireless communication circuit 218 may be operatively coupled to the processor 204 and may be configured to wirelessly communicate with one or more peripheral devices or other medical devices, such as an insulin pump or smart insulin pen.

In the example of FIG. 2, the medical device system 200 also includes optional external devices including, for example, a peripheral device 250. The peripheral device 250 may be any suitable user computing device such as, for example, a wearable device (e.g., activity monitor), such as a wearable device 120. In other examples, the peripheral device 250 may be a hand-held smart device (e.g., smartphone or other device such as a proprietary handheld device available from Dexcom), a tablet 114, a smart pen 116, or special-purpose computer 118 shown in FIG. 1.

The peripheral device 250 may include a UI 252, a memory circuit 254, a processor 256, a wireless communication circuit 258, a sensor 260, or any combination thereof. The peripheral device 250 may not necessarily include all the components shown in FIG. 2. The peripheral device 250 may also include a power source, such as a battery.

The UI 252 may, for example, be provided using any suitable input/output device or devices of the peripheral device 250 such as, for example, a touch-screen interface, a microphone (e.g., to receive voice commands), or a speaker, a vibration circuit, or any combination thereof. The UI 252 may receive information from the host or another user (e.g., instructions, glucose values). The UI 252 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 256 may be configured to present information to a user, or receive input from a user, via the UI 252. The processor 256 may also be configured to store and retrieve information, such as communication information (e.g., pairing information or data center access information), user information, sensor data or trends, or other information in the memory circuit 254. The wireless communication circuit 258 may include a transceiver and antenna configured to communicate via a wireless protocol, such as any of the wireless protocols described herein. The sensor 260 may, for example, include an accelerometer, a temperature sensor, a location sensor, biometric sensor, or blood glucose sensor, blood pressure sensor, heart rate sensor, respiration sensor, or another physiologic sensor.

The peripheral device 250 may be configured to receive and display sensor information that may be transmitted by sensor electronics 106 (e.g., in a customized data package that is transmitted to the display devices based on their respective preferences). Sensor information (e.g., blood glucose concentration level) or an alert or notification (e.g., “high glucose level”, “low glucose level” or “fall rate alert” may be communicated via the UI 252 (e.g., via visual display, sound, or vibration). In some examples, the peripheral device 250 may be configured to display or otherwise communicate the sensor information as it is communicated from the sensor electronics 106 (e.g., in a data package that is transmitted to respective display devices). For example, the peripheral device 250 may transmit data that has been processed (e.g., an estimated analyte value level that may be determined by processing raw sensor data), so that a device that receives the data may not be required to further process the data to determine usable information (such as the estimated analyte value level). In other examples, the peripheral device 250 may process or interpret the received information (e.g., to declare an alert based on glucose values or a glucose trend). In various examples, the peripheral device 250 may receive information directly from sensor electronics 106, or over a network (e.g., via a cellular or Wi-Fi network that receives information from the sensor electronics 106 or from a device that is communicatively coupled to the sensor electronics 106).

In the example of FIG. 2, the medical device system 200 includes an optional medical device 270. For example, the medical device 270 may be an external device used in addition to or instead of the peripheral device 250. The medical device 270 may be or include any suitable type of medical or other computing device including, for example, the medical device 108, peripheral medical device 122, wearable device 120, wearable sensor 130, or wearable sensor 136 shown in FIG. 1. The medical device 270 may include a UI 272, a memory circuit 274, a processor 276, a wireless communication circuit 278, a sensor 280, a therapy circuit 282, or any combination thereof.

Similar to the UI 252, the UI 272 may be provided using any suitable input/output device or devices of the medical device 270 such as, for example, a touch-screen interface, a microphone, or a speaker, a vibration circuit, or any combination thereof. The UI 272 may receive information from the host or another user (e.g., glucose values, alert preferences, calibration coding). The UI 272 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 276 may be configured to present information to a user, or receive input from a user, via the UI 272. The processor 276 may also be configured to store and retrieve information, such as communication information (e.g., pairing information or data center access information), user information, sensor data or trends, or other information in the memory circuit 274. The wireless communication circuit 278 may include a transceiver and antenna configured communicate via a wireless protocol, such as any of the wireless protocols described herein.

The sensor 280 may, for example, include an accelerometer, a temperature sensor, a location sensor, biometric sensor, or blood glucose sensor, blood pressure sensor, heart rate sensor, respiration sensor, or another physiologic sensor. The medical device 270 may include two or more sensors (or memories or other components), even though only one sensor 280 is shown in the example in FIG. 2. In various examples, the medical device 270 may be a smart handheld glucose sensor (e.g., blood glucose meter), drug pump (e.g., insulin pump), or other physiologic sensor device, therapy device, or combination thereof.

In examples where medical device 270 is or includes an insulin pump, the pump and analyte sensor system 102 may be in two-way communication (e.g., so the pump can request a change to an analyte transmission protocol, e.g., request a data point or request data on a more frequent schedule), or the pump and analyte sensor system 102 may communicate using one-way communication (e.g., the pump may receive analyte concentration level information from the analyte sensor system). In one-way communication, a glucose value may be incorporated in an advertisement message, which may be encrypted with a previously shared key. In a two-way communication, a pump may request a value, which the analyte sensor system 102 may share, or obtain and share, in response to the request from the pump, and any or all of these communications may be encrypted using one or more previously shared keys. An insulin pump may receive and track analyte (e.g., glucose) values transmitted from analyte sensor system 102 using one-way communication to the pump for one or more of a variety of reasons. For example, an insulin pump may suspend or activate insulin administration based on a glucose value being below or above a threshold value.

In some examples, the medical device system 200 includes two or more peripheral devices and/or medical devices that each receive information directly or indirectly from the analyte sensor system 102. Because different display devices provide many different user interfaces, the content of the data packages (e.g., amount, format, and/or type of data to be displayed, alarms, and the like) may be customized (e.g., programmed differently by the manufacturer and/or by an end user) for each device. For example, referring now to the example of FIG. 1, a plurality of different peripheral devices may be in direct wireless communication with sensor electronics 106 (e.g., such as an on-skin sensor electronics 106 that are physically connected to the continuous analyte sensor 104) during a sensor session to enable a plurality of different types and/or levels of display and/or functionality associated with the displayable sensor information, or, to save battery power in the sensor system 102, one or more specified devices may communicate with the analyte sensor system 102 and relay (i.e., share) information to other devices directly or through a server system (e.g., a network-connected data center) 126.

FIG. 3 is a side view of an example analyte sensor 334 that may be implanted into a host. An enclosure 302 may be adhered to the host's skin using an adhesive pad 308. The adhesive pad 308 may be formed from an extensible material, which may be removably attached to the skin using an adhesive. Sensor electronics may be positioned within the enclosure 302. The sensor 334 may extend from the enclosure 302 and under the skin of a host, as shown.

FIG. 4 is a side view of another example analyte sensor 434 in an arrangement including a mounting unit 414 and an electronics unit 418. The mounting unit 414 may be adhered to the host's skin using an adhesive pad 408, which may be like the adhesive pad 308 described herein. The electronics unit 418 comprises an enclosure 402 that may have sensor electronics positioned thereon. In some examples, the electronics unit 418 and mounting unit 414 are arranged in a manner like the sensor electronics 106 and sensor mounting unit 290 shown in FIGS. 1 and 2. For example, the sensor 434 may extend from the enclosure 402 via the mounting unit 414.

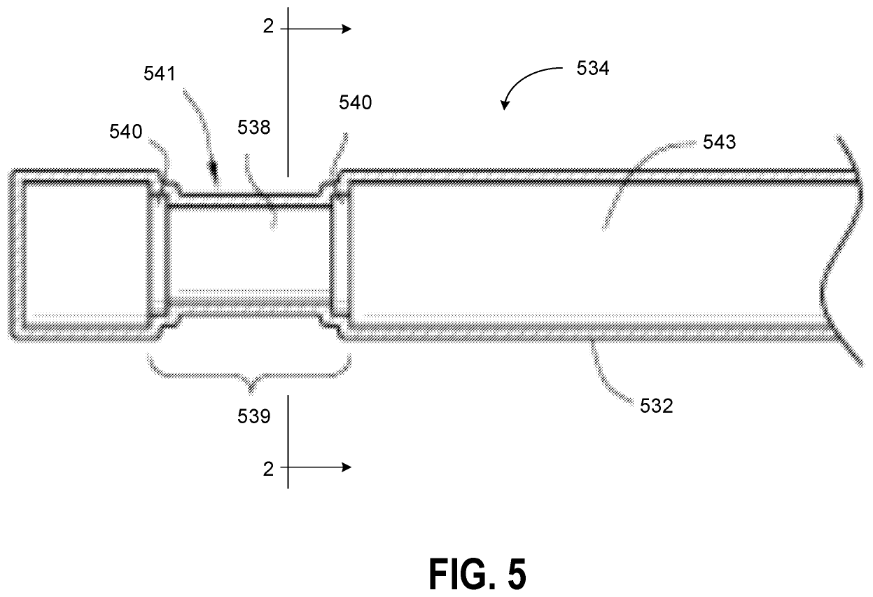

FIG. 5 is an enlarged view of a distal portion of an analyte sensor 534. The analyte sensor 534 illustrates one example arrangement that may be used to implement the analyte sensors described herein, such as, for example, the analyte sensors 104, 334, 434. The analyte sensor 534 may be adapted for insertion under the host's skin and may be mechanically coupled to an enclosure, such as the enclosures 402, and/or to a mounting unit 414, such as the mounting unit 414. The analyte sensor 534 may be electrically coupled to sensor electronics, which may be positioned within the enclosure 302, 402.

The example analyte sensor 534 shown in FIG. 5 includes an elongated conductive body 541. The elongated conductive body 541 can include a core with various layers positioned thereon. A first layer 538 that at least partially surrounds the core and includes a working electrode, for example located in window 539). In some examples, the core and the first layer 538 are made of a single material (such as, for example, platinum). In some examples, the elongated conductive body 541 is a composite of two conductive materials, or a composite of at least one conductive material and at least one non-conductive material. A membrane system 532 is located over the working electrode and may cover other layers and/or electrodes of the sensor 534, as described herein.

The first layer 538 may be formed of a conductive material. The working electrode (at window 539) is an exposed portion of the surface of the first layer 538. Accordingly, the first layer 538 is formed of a material configured to provide a suitable electroactive surface for the working electrode. Examples of suitable materials include, but are not limited to, platinum, platinum-iridium, gold, palladium, iridium, graphite, carbon, a conductive polymer, an alloy, and/or the like.

A second layer 540 surrounds at least a portion of the first layer 538, thereby defining boundaries of the working electrode. In some examples, the second layer 540 serves as an insulator and is formed of an insulating material, such as polyimide, polyurethane, parylene, or any other suitable insulating materials or materials. In some examples, the second layer 540 is configured such that the working electrode (of the layer 538) is exposed via the window 539.

In some examples, the sensor 534 further includes a third layer 543 comprising a conductive material. The third layer 543 may comprise a reference electrode. In some examples, the third layer 543, including the reference electrode, is formed of a silver-containing material that is applied onto the second layer 540 (e.g., an insulator). The silver-containing material may include various materials and be in various forms such as, for example, Ag/AgCl-polymer pasts, paints, polymer-based conducting mixtures, inks, etc.

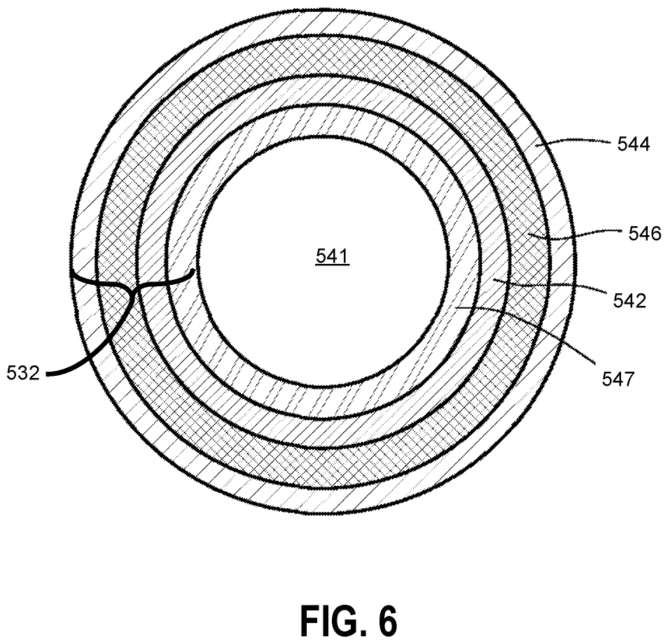

The analyte sensor 534 may include two (or more) electrodes, e.g., a working electrode at the layer 538 and exposed at window 539 and at least one additional electrode, such as a reference electrode of the layer 543. In the example arrangement of FIGS. 5-6, the reference electrode also functions as a counter electrode, although other arrangements can include a separate counter electrode. While the analyte sensor 534 may be used with a mounting unit in some examples, in other examples, the analyte sensor 534 may be used with other types of sensor systems. For example, the analyte sensor 534 may be part of a system that includes a battery and sensor in a single package, and may optionally include, for example, a near-field communication (NFC) circuit.

FIG. 6 is a cross-sectional view through the sensor 534 of FIG. 5 on plane 2-2 illustrating a membrane system 532. The membrane system 532 may include a number of domains (e.g., layers). In an example, the membrane system 532 may include an enzyme domain 542, a diffusion resistance domain 544, and a bioprotective domain 546 located around the working electrode. In some examples, a unitary diffusion resistance domain and bioprotective domain may be included in the membrane system 532 (e.g., wherein the functionality of both the diffusion resistance domain and bioprotective domain are incorporated into one domain).

The membrane system 532, in some examples, also includes an electrode layer 547. The electrode layer 547 may be arranged to provide an environment between the surfaces of the working electrode and the reference electrode that facilitates the electrochemical reaction between the electrodes. For example, the electrode layer 547 may include a coating that maintains a layer of water at the electrochemically reactive surfaces of the sensor 534.

In some examples, the sensor 534 may be configured for short-term implantation (e.g., from about 1 to 30 days). However, it is understood that the membrane system 532 can be modified for use in other devices, for example, by including only one or more of the domains, or additional domains. For example, a membrane system 532 may include a plurality of resistance layers, or a plurality of enzyme layers. In some examples, the resistance domain 544 may include a plurality of resistance layers, or the enzyme domain 542 may include a plurality of enzyme layers.

The diffusion resistance domain 544 may include a semipermeable membrane that controls the flux of oxygen and glucose to the underlying enzyme domain 542. As a result, the upper limit of linearity of glucose measurement is extended to a much higher value than that which is achieved without the diffusion resistance domain 544.

In some examples, the membrane system 532 may include a bioprotective domain 546, also referred to as a domain or biointerface domain, comprising a base polymer. However, the membrane system 532 of some examples can also include a plurality of domains or layers including, for example, an electrode domain, an interference domain, or a cell disruptive domain, such as described in more detail elsewhere herein and in U.S. Pat. Nos. 7,494,465, 8,682,608, and 9,044,199, which are incorporated herein by reference in their entirety.

It is to be understood that sensing membranes modified for other sensors, for example, may include fewer or additional layers. For example, in some examples, the membrane system 532 may comprise one electrode layer, one enzyme layer, and two bioprotective layers, but in other examples, the membrane system 532 may comprise one electrode layer, two enzyme layers, and one bioprotective layer. In some examples, the bioprotective layer may be configured to function as the diffusion resistance domain 544 and control the flux of the analyte (e.g., glucose) to the underlying membrane layers.

In some examples, one or more domains of the sensing membranes may be formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers.

In some examples, the sensing membrane can be deposited on the electroactive surfaces of the electrode material using known thin or thick film techniques (for example, spraying, electro-depositing, dipping, or the like). The sensing membrane located over the working electrode does not have to have the same structure as the sensing membrane located over the reference electrode; for example, the enzyme domain 542 deposited over the working electrode does not necessarily need to be deposited over the reference or counter electrodes.

Although the examples illustrated in FIGS. 5-6 involve circumferentially extending membrane systems, the membranes described herein may be applied to any planar or non-planar surface, for example, the substrate-based sensor structure of U.S. Pat. No. 6,565,509 to Say et al., which is incorporated by reference.

In an example in which the analyte sensor 534 is a glucose sensor, glucose analyte can be detected utilizing glucose oxidase. Glucose oxidase reacts with glucose to produce hydrogen peroxide (H₂O₂). The hydrogen peroxide reacts with the surface of the working electrode, producing two protons (2H⁺), two electrons (2e⁻) and one molecule of oxygen (O₂). This produces an electronic current that may be detected by the sensor electronics 106. The amount of current is a function of the glucose concentration level. A calibration curve may be used to provide an estimated glucose concentration level based on a measured current. The amount of current is also a function of the diffusivity of glucose through the sensor membrane. The glucose diffusivity may change over time, which may cause the sensor glucose sensitivity to change over time, or “drift.”

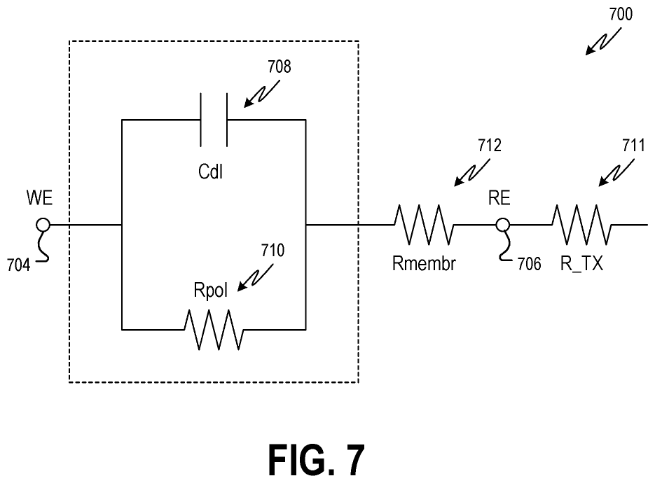

FIG. 7 is a schematic illustration of a circuit 700 that represents the behavior of an example analyte sensor, such as the analyte sensor 534 shown in FIGS. 5-6. As described above, the interaction of hydrogen peroxide (generated from the interaction between glucose analyte and glucose oxidase) and working electrode (WE) 704 produces a voltage differential between the working electrode (WE) 704 and reference electrode (RE) 706 which drives a current. The current may make up all or part of a raw sensor signal that is measured by sensor electronics, such as the sensor electronics 106 of FIGS. 1-2, and used to estimate an analyte concentration (e.g., glucose concentration).

The circuit 700 also includes a double-layer capacitance (Cdl) 708, which occurs at an interface between the working electrode (WE) 704 and the adjacent membrane (not shown in FIG. 7, see, e.g., FIGS. 5-6 above). The double-layer capacitance (Cdl) may occur at an interface between the working electrode 704 and the adjacent membrane due to the presence of two layers of ions with opposing polarity, as may occur during application of an applied voltage between the working electrode 704 and reference electrode. The equivalent circuit 700 may also include a polarization resistance (Rpol) 710, which may be relatively large, and may be modeled, for example, as a static value (e.g., 100 mega-Ohms), or as a variable quantity that varies as a function of glucose concentration level.

An estimated analyte value may be determined from a raw sensor signal based upon a measured current (or charge flow) through the analyte sensor membrane 712 when a bias potential is applied to the sensor circuit 700. For example, sensor electronics or another suitable computing device can use the raw sensor signal and a sensitivity of the sensor, which correlates a detected current flow to a glucose concentration level, to generate the estimated analyte value. In some examples, the device also uses a break-in characteristic, as described herein.

With reference to the equivalent circuit 700, when a voltage is applied across the working and reference electrodes 704 and 706, a current may be considered to flow (forward or backward depending on polarity) through the internal electronics of transmitter (represented by R Tx internal) 711; through the reference electrode (RE) 706 and working electrode (WE) 704, which may be designed to have a relatively low resistance; and through the sensor membrane 712 (Rmembr, which is relatively small). Depending on the state of the circuit, current may also flow through, or into, the relatively large polarization resistance 710 (which is indicated as a fixed resistance but may also be a variable resistance that varies with the body's glucose level, where a higher glucose level provides a smaller polarization resistance), or into the double-layer capacitance 708 (i.e., to charge the double-layer membrane capacitor formed at the working electrode 704), or both.

The impedance (or conductance) of the membrane (Rmembr) 712 is related to electrolyte mobility in the membrane, which is in turn related to glucose diffusivity in the membrane. As the impedance goes down (i.e., conductance goes up, as electrolyte mobility in the membrane 712 goes up), the glucose sensitivity goes up (i.e., a higher glucose sensitivity means that a particular glucose concentration will produce a larger signal in the form of more current or charge flow). Impedance, glucose diffusivity, and glucose sensitivity are further described in U.S. Patent Publication No. US2012/0262298, which is incorporated by reference in its entirety.

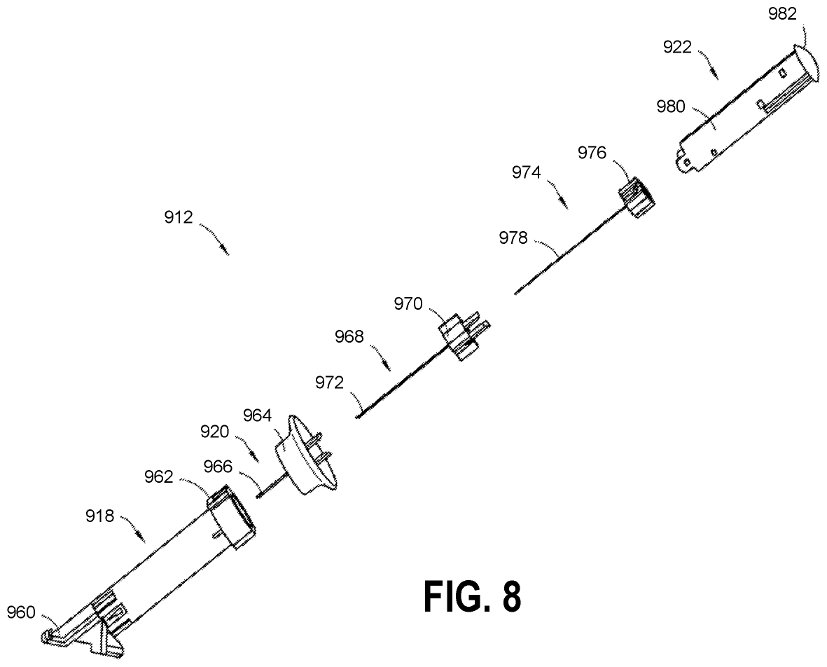

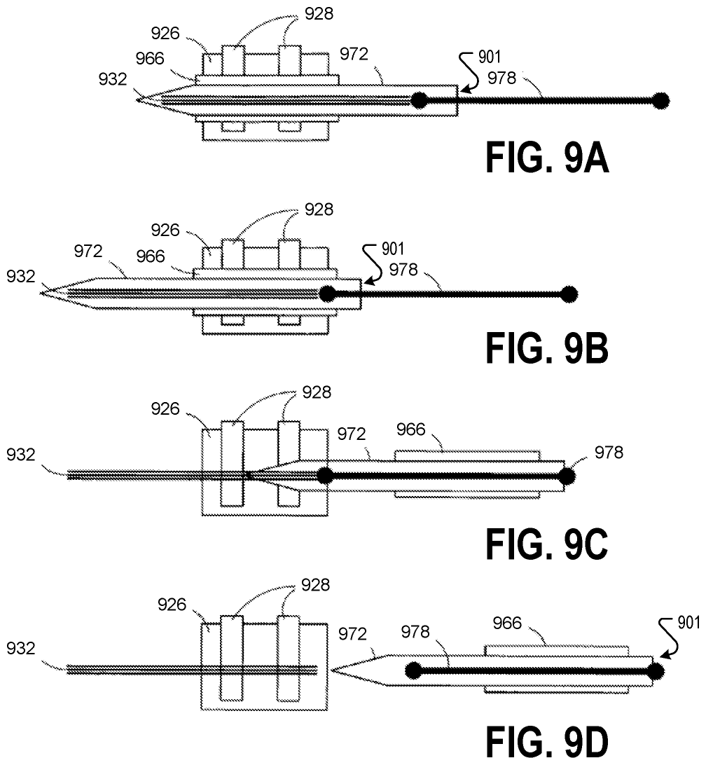

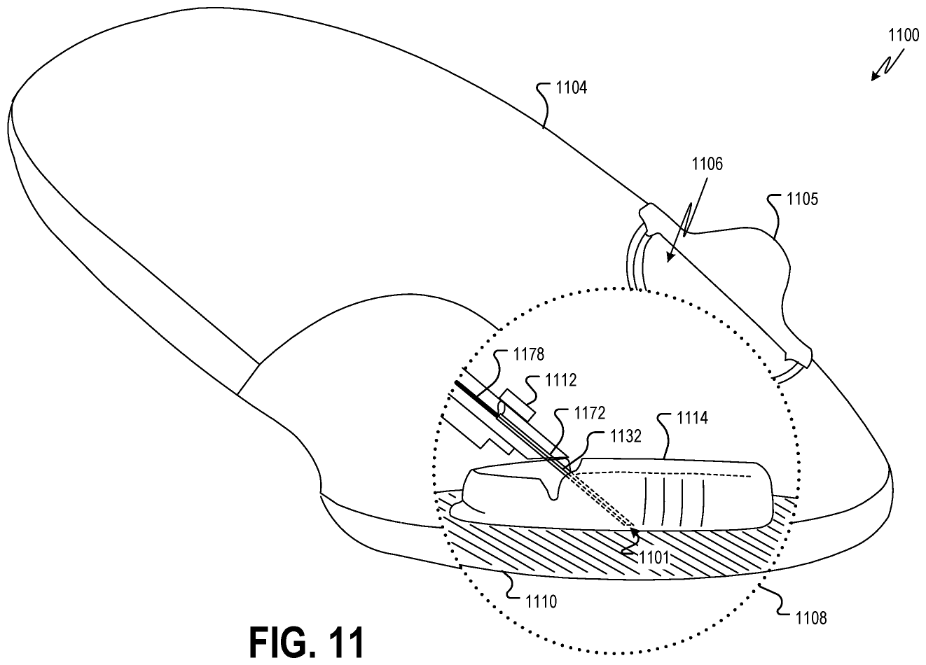

In various examples, an analyte sensor is packaged with a sensor applicator. FIGS. 8, 9A-D, 10 and 11 show example implementations of a sensor applicator. FIG. 8 is an exploded side view showing one example of a sensor applicator 912. In this example, the sensor applicator 912 includes an applicator body 918 that aides in aligning and guiding the sensor applicator components. The applicator body 918 includes an applicator body base 960 that matingly engages the mounting unit 914 (FIG. 11) and an applicator body cap 962 that enables appropriate relationships (for example, stops) between the sensor applicator components.

A guide tube subassembly 920 includes a guide tube carrier 964 and a guide tube 966. In some examples, the guide tube is a cannula. The guide tube carrier 964 slides along the applicator body 918 and maintains the appropriate relative position of the guide tube 966 during insertion and subsequent retraction. For example, prior to and during insertion of the sensor, the guide tube 966 extends through the contact subassembly 926 to maintain an opening that enables easy insertion of the needle therethrough (see FIGS. 9A to 9D). During retraction of the sensor, the guide tube subassembly 920 is pulled back, engaging with, and causing the needle and associated moving components to retract back into the sensor applicator 912 (See FIGS. 9C and 9D). In some examples, a lubricant (e.g., petroleum jelly) is placed within the contact subassembly 926 such that it surrounds the guide tube 966 (e.g., cannula), thereby allowing the guide tube 966 to easily retract back into the sensor applicator 912, for example, without causing compression or deformation in the contact subassembly 926.

A needle subassembly 968 is provided that includes a needle carrier 970 and needle 972. The needle carrier 970 cooperates with the other sensor applicator components and carries the needle 972 between its extended and retracted positions. The needle 972 can be of any appropriate size that can encompass an analyte sensor 932 (FIGS. 9A-9D) and aid in its insertion into the host. Example sizes include from about 32 gauge or less to about 18 gauge or more, more preferably from about 28 gauge to about 25 gauge, to provide a comfortable insertion for the host. Referring to the inner diameter of the needle, approximately 0.006 inches to approximately 0.023 inches is may be used, and 0.013 inches may also be used. The needle carrier 970 is configured to engage with the guide tube carrier 964, while the needle 972 is configured to slidably nest within the guide tube 966, which allows for easy guided insertion (and retraction) of the needle through the contact subassembly 926.

A push rod subassembly 974 is provided that includes a push rod carrier 976 and a push rod 978. The push rod carrier 976 cooperates with other sensor applicator components to ensure that the analyte sensor 932 is properly inserted into the host's skin, namely the push rod carrier 976 carries the push rod 978 between its extended and retracted positions. In this embodiment, the push rod 978 is configured to slidably nest within a cannula 901 of the needle 972, which allows for the analyte sensor 932 to be pushed (released) from the needle 972 upon retraction of the needle 972. This is described in more detail with reference to FIGS. 9A-9D. In some examples, a slight bend or serpentine shape is designed into or allowed in the sensor in order to maintain the sensor within the needle 972 by interference. While not wishing to be bound by theory, it is believed that a slight friction fit of the analyte sensor 932 within the needle 972 may minimize motion of the analyte sensor 932 during withdrawal of the needle 972 and maintains the analyte sensor 932 within the needle prior to withdrawal of the needle 972.

A plunger subassembly 922 is provided that includes a plunger 980 and plunger cap 982. The plunger subassembly 922 cooperates with other sensor applicator components to ensure proper insertion and subsequent retraction of the needle 972. In this example, the plunger 980 is configured to engage with the push rod 978 to ensure the analyte sensor 932 remains extended (namely, in the host) during retraction, such as is described in more detail with reference to FIG. 9C.

FIGS. 9A through 9D are schematic side cross-sectional views that illustrate the applicator components and their cooperating relationships at various stages of sensor insertion. FIG. 9A shows one example of the needle 972 and analyte sensor 932 loaded prior to sensor insertion. FIG. 9B shows one example of the needle 972 and analyte sensor 932 after sensor insertion. FIG. 9C shows one example of the analyte sensor 932 and needle 972 during needle retraction. FIG. 9D shows one example of the analyte sensor 932 remaining within the contact subassembly 926 after needle retraction. Although the examples of FIGS. 8, 9A-9D and 11 suggest manual insertion and/or retraction of the various components, automation of one or more of the stages can also be employed. For example, spring-loaded mechanisms that can be triggered to automatically insert and/or retract the sensor, needle, or other cooperative applicator components can be implemented.

Referring to FIG. 9A, the analyte sensor 932 is shown disposed within the needle 972, which is disposed within the guide tube 966. In this example, the guide tube 966 is provided to maintain an opening within the contact subassembly 926 and/or contacts 928 to provide minimal friction between the needle 972 and the contact subassembly 926 and/or contacts 928 during insertion and retraction of the needle 972. However, the guide tube 966 is an optional component, which can be advantageous in some examples where the contact subassembly 926 and/or the contacts 928 are formed from an elastomer or other material with a relatively high friction coefficient. The guide tube 966 can be omitted, for example, in other examples in which the contact subassembly 926 and/or the contacts 928 are formed from a material with a relatively low friction coefficient (for example, hard plastic or metal). A guide tube 966, or the like, may be advantageous in examples in which the contact subassembly 926 and/or the contacts 928 are formed from a material designed to frictionally hold the analyte sensor 932 (see FIG. 9D), for example, by the relaxing characteristics of an elastomer, or the like. In these examples, the guide tube 966 may be provided to ease insertion of the needle 972 through the contacts 928, while allowing for a frictional hold of the contacts 928 on the analyte sensor 932 upon subsequent needle retraction. Stabilization of the analyte sensor 932 in or on the contacts 928 is described in more detail with reference to FIG. 9D. Although FIG. 9A illustrates the needle 972 and analyte sensor 932 inserted into the contacts subassembly 926 as the initial loaded configuration, alternative embodiments contemplate a step of loading the needle 972 through the guide tube 966 and/or contacts 928 prior to sensor insertion.

Referring to FIG. 9B, the analyte sensor 932 and needle 972 are shown in an extended position. In this stage, the push rod 978 has been forced to a forward position, for example by pushing on the plunger shown in FIG. 7, or the like. The plunger 980 (FIG. 9) is designed to cooperate with other of the sensor applicator components to ensure that analyte sensor 932 and the needle 972 extend together to a forward position (as shown). For example, the push rod 978 may be designed to cooperate with other of the sensor applicator components to ensure that the analyte sensor 932 maintains the forward position simultaneously within the needle 972.

Referring to FIG. 9C, the needle 972 is shown during the retraction process. In this stage, the push rod 978 is held in its extended (forward) position in order to maintain the analyte sensor 932 in its extended (forward) position until the needle 972 has substantially fully retracted from the contacts 928. Simultaneously, the cooperating sensor applicator components retract the needle 972 and guide tube 966 backward by a pulling motion (manual or automated) thereon. In preferred embodiments, the guide tube carrier 964 (FIG. 9) engages with cooperating applicator components such that a backward (retraction) motion applied to the guide tube carrier retracts the needle 972 and guide tube 966, without (initially) retracting the push rod 978. In an alternative embodiment, the push rod 978 can be omitted and the analyte sensor 932 held it its forward position by a cam, elastomer, or the like, which is in contact with a portion of the sensor while the needle moves over another portion of the sensor. One or more slots can be cut in the needle to maintain contact with the sensor during needle retraction.

Referring to FIG. 9D, the needle 972, guide tube 966, and push rod 978 are all retracted from contact subassembly 926, leaving the analyte sensor 932 disposed therein. The cooperating sensor applicator components are designed such that when the needle 972 has substantially cleared from the contacts 928 and/or contact subassembly 926, the push rod 978 is retracted along with the needle 972 and guide tube 966. The sensor applicator 912 can then be released (manually or automatically) from the contacts 928.

In various examples, the contacts 928 are elastomeric contacts to ensure a retention force that retains the analyte sensor 932 within the mounting unit and to ensure stable electrical connection of the analyte sensor 932 and its associated contacts 928. Although the illustrated embodiments and associated text describe the analyte sensor 932 extending through the contacts 928 to form a friction fit therein, a variety of alternatives are contemplated. In some examples, the sensor 932 is configured to be disposed adjacent to the contacts 928 (rather than between the contacts 928). The contacts 928 can be constructed in a variety of known configurations, for example, metallic contacts, cantilevered fingers, pogo pins, or the like, which are configured to press against the sensor after needle retraction.

The illustrated embodiments are designed with coaxial contacts 928; namely, the contacts 928 are configured to contact the working and reference electrodes of the analyte sensor 932 axially along a distal portion of the analyte sensor 932. For example, the working electrode of the analyte sensor 932 may extend farther than the reference electrode, which allows coaxial connection of the electrodes with the contacts 928 at locations spaced along the distal portion of the sensor.

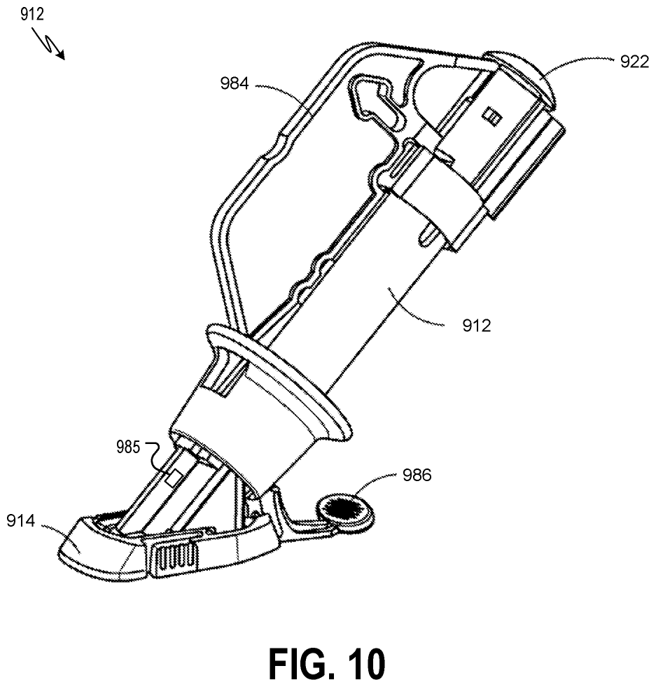

FIG. 10 is a perspective view of a sensor applicator 912 and mounting unit 914 according to one example including a safety latch mechanism 984. Although FIG. 10 shows the sensor applicator 912 engaged with a sensor mounting unit 914, it will be appreciated that the sensor applicator 912 may be configured to engage instead with an enclosure, such as the enclosure 302 described with respect to FIG. 3.

In the example of FIG. 10, the safety latch mechanism 984 is configured to lock the plunger subassembly 922 in a stationary position such that it cannot be accidentally pushed prior to release of the safety latch mechanism 984. In this example, the analyte sensor 932 is preferably packaged (e.g., shipped) in this locked configuration, where the safety latch mechanism 984 holds the plunger subassembly 922 in its extended position. This may prevent the analyte sensor 932 from being prematurely inserted (e.g., accidentally released). The safety latch mechanism 984 may be configured such that a pulling force shown in the direction of the arrow (see FIG. 10) releases the lock of the safety latch mechanism 984 on the plunger subassembly 922, thereby allowing sensor insertion. Although one safety latch mechanism 984 that locks the plunger subassembly 922 is illustrated and described herein, a variety of safety latch mechanism configurations that lock the sensor to prevent it from prematurely releasing (i.e., that lock the sensor prior to release of the safety latch mechanism) are contemplated, as can be appreciated by one skilled in the art and fall within the scope of the preferred embodiments.

FIG. 10 additionally illustrates a force-locking mechanism 986 included in certain alternative embodiments of the sensor system, wherein the force-locking mechanism 986 is configured to ensure a proper mate between an electronics unit (e.g., electronics unit 418 of FIG. 4) and the mounting unit 914. In some circumstances, it can be advantageous to ensure the electronics unit has been properly mated (e.g., snap-fit or sealingly mated) to the mounting unit. Accordingly, upon release of the sensor applicator 912 from the mounting unit 914 after sensor insertion, the force-locking mechanism 986 allows the user to ensure a proper mate and/or seal therebetween.

In practice, a user pivots (e.g., lifts or twists) the force-locking mechanism such that it provides force on the electronics unit (such as electronics unit 418 of FIG. 4) by pulling up on the circular tab illustrated in FIG. 10. The force-locking mechanism is preferably released thereafter. Although one system and one method for providing a secure and/or sealing fit between the electronics unit and the mounting unit are illustrated, various other force-locking mechanisms can be employed that utilize a variety of systems and methods for providing a secure and/or sealing fit between the electronics unit and the mounting unit (housing).

In some examples, the sensor applicator 912 shown in FIGS. 8, 9A-9D and 10 can also comprise a transmitter 985. The transmitter 985 is configured to generate a wireless signal that may be detected by the sensor electronics 106, as described herein. The wireless signal generated by the transmitter 985 may be in a first state or undeployed state before the sensor applicator 912 inserts the sensor. After and/or as the sensor is inserted, the transmitter 985 may modify the wireless signal to a second state or deployed state. The sensor electronics may detect the change in state of the wireless signal to determine whether to transition from a sleep mode to an active mode, as described herein.

The transmitter 985 may be of any suitable design that generates a wireless signal having at least two states. In some examples, the transmitter 985 is an electromagnet or permanent magnet and the wireless signal is a magnetic field generated by the transmitter 985. One state of the wireless signal may be the presence of the magnetic field (e.g., the magnetic field having a field strength greater than a threshold). Another state of the wireless signal may be the absence of the magnetic field (e.g., the magnetic field having a field strength less than a threshold). The presence of the magnetic field may indicate the undeployed state and the absence of the magnetic field may indicate the deployed state, or visa versa.

In some examples in which the transmitter 985 includes an electromagnet and/or permanent magnet, the first state of the wireless signal may correspond to a first magnetic polarity of the magnetic field and the second state of the wireless signal may correspond to a second magnetic polarity of the magnetic field, which may be opposite the first polarity. One polarity of the magnetic field may correspond to the undeployed state and another polarity of the magnetic field may correspond to the deployed state. The transmitter 985 may transition the wireless signal from the first state to the second state, for example, by reversing the direction of current provided to an electromagnet and/or reversing the physical orientation of the magnet and/or the like.

Other suitable transmitter arrangements may also be used to modify the state of the wireless signal in other ways. In some examples, the transmitter 985 is or includes an optical transmitter configured to generate an optical signal. Also, in some examples, the transmitter 985 is or includes a sonic transmitter configured to generate a sonic signal. In some examples, the transmitter 985 is or includes a radio frequency transmitter, such as an RFID transmitter.

FIG. 11 is a diagram showing another example of an analyte sensor applicator 1100. In the example of FIG. 11, the sensor applicator 1100 includes an applicator enclosure 1104. The sensor applicator 1100 is automated and, for example, includes a spring-loaded mechanism for initiating sensor insertion (not shown in FIG. 11). For example, the spring-loaded mechanism may be actuated by depressing an insertion button 1106. A safety latch mechanism 1105 is positioned over the insertion button 1106 to prevent accidental insertion of the analyte sensor 1132. The safety latch mechanism 1105 can be removed, for example, by pulling it away from the applicator enclosure 1104.

FIG. 11 includes a window 1108 showing components that are inside of the applicator enclosure 1104 including, for example, a needle 1172 and a mounting unit 1114. The mounting unit 1114 is coupled to an example adhesive pad 1110 for adhering the mounting unit 1114 to the skin of a host. In examples that do not include a separate electronics unit, such as the example of FIG. 3, the enclosure 302 and associated adhesive pad 308 may be positioned in a manner similar to that of the mounting unit 1114 and adhesive pad 1110.

In the example of FIG. 11, a push rod 1178 may operate in a manner similar to that of the push rod 978 described above to push the analyte sensor 1132 into the host. A lumen 1101 of the needle 1172 can include a hydrating agent, for example, as described herein. The example of FIG. 11, the applicator 1100 also includes a transmitter 1112 which may be arranged, for example, in a manner similar to that of the transmitter 985 of FIG. 10.

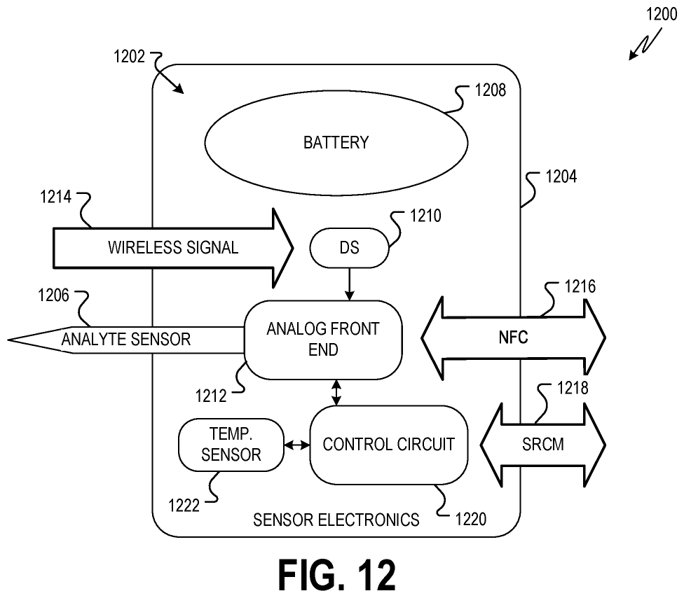

FIG. 12 is a diagram showing one example of an analyte sensor system 1200 including an analyte sensor 1206 and sensor electronics 1202. The analyte sensor 1206 may be arranged, for example, as described herein with respect to FIGS. 3-6 and/or according to any other suitable arrangement such as, for example a planar sensor arrangement.

The sensor electronics 1202 are positioned within an enclosure 1204. The analyte sensor 1206 extends from the enclosure 1204 as shown. In some examples, the analyte sensor 1206 is mechanically coupled to the enclosure 1204, for example, as illustrated in FIG. 3. In some examples, the analyte sensor 1206 is mechanically coupled to a sensor mounting unit, as described herein.

The analyte sensor 1206 may be in electrical communication with an analog front end 1212 of the sensor electronics 1202. In examples in which the analyte sensor 1206 is mechanically coupled to the enclosure 1204, the analyte sensor 1206 may extend through the enclosure 1204 in a sealed manner and be in direct electrical contact with the analog front end 1212. In examples in which the analyte sensor 1206 is mechanically coupled to a sensor mounting unit, an electrical connection between the analyte sensor 1206 and the analog front end 1212 may be via a connector between the sensor mounting unit and the enclosure 1204.

The analog front end 1212 may be configured to receive analog electrical signals from various components of the sensor electronics 1202 and provide corresponding digital electrical signals to a control circuit 1220. For example, as described, the analyte sensor 1206 may be in electrical communication with the analog front end 1212 to provide an analog raw sensor signal to the analog front end 1212. The analog front end 1212 may comprise various amplifiers, filters, conditioners, analog-to-digital converters, and/or the like to condition the raw sensor signal and covert it to a digital raw sensor signal, which may be provided to the control circuit 1220. The control circuit 1220 may be configured to convert the digital raw sensor signal to an estimated analyte value, which may be output as described herein.

The analog front end 1212 may also be in communication with a deployment sensor 1210. The deployment sensor 1210 receives a wireless signal 1214. The wireless signal 1214 may be generated by a transmitter at an applicator, such as, for example, the transmitter 1112 of the applicator 1100 and/or the transmitter 985 of the applicator 912. The deployment sensor 1210 may be configured to detect the wireless signal 1214 and generate an output indicative of the state of the wireless signal 1214. In examples where the wireless signal 1214 is a magnetic signal, the deployment sensor 1210 may be or comprise a magnetic sensor, such as a Tunnel Magento-Resistance effect (TMR) sensor. In examples where the wireless signal 1214 is an optical signal, the deployment sensor 1210 may be or comprise a photoresistor or another optical sensor. In examples where the wireless signal 1214 is a radio frequency (RF) or other similar signal, the deployment sensor 1210 may comprise an antenna and/or other RF receiver components. The analog front end 1212 may receive an output of the deployment sensor 1210 and provide the output to the control circuit 1220 for further processing. In some examples, as shown in FIG. 12, the analog front end 1212 and control circuit 1220 may be coupled to the analyte sensor 1206 prior to deployment. In this way, the analog front end 1212 and control circuit 1220 may detect deployment of the analyte sensor 1206 (e.g., via wireless signal 1214) close in time to the actual insertion of the analyte sensor 1206.

In the example of FIG. 12, the analog front end 1212 comprises circuitry for receiving and transmitting near field communication (NFC) signals 1216. NFC signals 1216 may be utilized to communicate with various external devices. In some examples, the wireless signal 1214 may be implemented as an NFC signal 1216.

The control circuit 1220 is configured to receive digital signals from the analog front end 1212 and perform various processing on the signals. For example, the control circuit 1220 may be and/or include a microcontroller or other processor. One or more processers of the control circuit 1220 may be programmed to execute software instructions for executing various operations, for example, as described herein. The software instructions may be stored at a data storage that may be part of the control circuit 1220 or may be implemented at a different location.

In some examples, the control circuit 1220 converts a raw sensor signal (e.g., a digital raw sensor signal) to a corresponding estimated analyte value. The control circuit 1220 may provide result data including the estimated analyte value as an output, for example, in the manner described herein. In the example of FIG. 12, the control circuit 1220 is in communication with a temperature sensor 1222, for example, as described herein. In this example, the temperature sensor 1222 is a digital sensor. In other examples, an analog temperature sensor (or other sensor, as described herein) may be in communication with the control circuit 1220 via the analog front end 1212. In some examples, the control circuit 1220 is also configured to communicate with one or more external devices via a short-range wireless communication medium 1218 such as, for example, Bluetooth®, Bluetooth LE® and/or the like.

The control circuit 1220 may also be programmed to transition the analyte sensor system 1200 from a sleep mode to an active mode. For example, various components of the sensor electronics 1202 are powered by a battery 1208. In some examples, the battery 1208 is installed at the time that the analyte sensor system 1200 is manufactured. In some examples, the analyte sensor system 1200 is configured to operate for its full lifecycle on a single charge of the battery 1208. Accordingly, the analyte sensor system 1200 may be configured in a sleep mode after manufacturing. In the sleep mode, the sensor electronics 1202 maintains the analyte sensor system 1200 in an arrangement for low power consumption. For example, the non-essential components of the analyte sensor system 1200 may be switched off. Also, for example, operations executed by the sensor electronics 1202 may be minimized to save power. The control circuit 1220 may be programmed to maintain the sleep mode until the analyte sensor 1206 is inserted in vivo into a host and a sensor session is to begin.

When the control circuit 1220 determines that a sensor session is to begin, the control circuit 1220 transitions the analyte sensor system 1200 into the active mode. This may include, for example, applying a bias condition to the analyte sensor, using the raw sensor signal (e.g., a digital raw sensor signal) to determine and output an estimated analyte concentration, and/or the like. The sensor electronics 1202 may determine to transition the analyte sensor system 1200 to the active mode, for example, using the wireless signal 1214 and/or the raw sensor signal provided by the analyte sensor 1206. Although specific circuitry, components, and interconnections are illustrated in the exemplary analyte sensor system 1200 shown in FIG. 12, alternative embodiments may incorporate additional, fewer, and/or alternative circuitry, components, and/or interconnections.

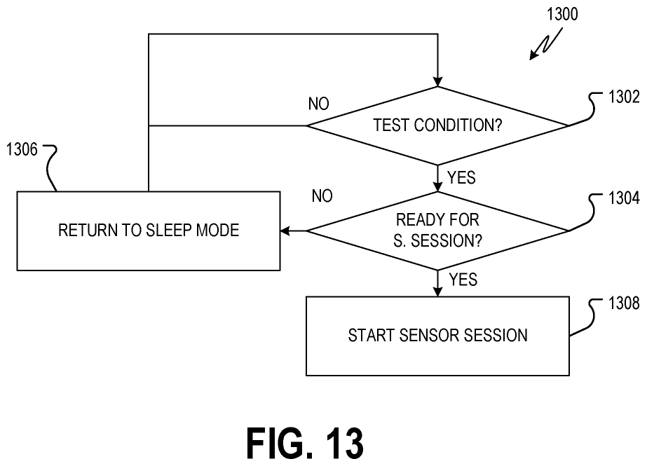

FIG. 13 is a flowchart showing one example of a process flow 1300 that may be executed by the sensor electronics of a sensor system to transition the sensor system from a sleep mode to an active mode. At operation 1302, the sensor electronics 1202 evaluates a test condition. The test condition is a condition that indicates a potential deployment or insertion of the analyte sensor 1206. Evaluating the test condition may include polling the deployment sensor 1210 and/or the analyte sensor 1206. For example, the test condition may be based on the raw sensor signal generated by the analyte sensor 1206 and/or on a signal received from the deployment sensor 1210.

In some examples, the test condition is based at least in part the raw sensor signal generated by the analyte sensor 1206. For example, the raw sensor signal may change after the analyte sensor 1206 is inserted into the host, e.g., under the skin as described herein. For example, prior to insertion, the analyte sensor 1206 may generate a small signal or no signal, as the analyte sensor system 1200 may be packed for storage and/or transportation such that negligible electrochemical activity takes place at the analyte sensor 1206. Upon insertion, however, the analyte sensor 1206 may begin to generate a non-negligible raw sensor signal as in vivo reactions begin to occur. Accordingly, the sensor electronics 1202 may execute the operation 1302 by polling the raw sensor signal and comparing it to a deploy test threshold. If the raw sensor signal is about the deploy test threshold, the test condition may be met. In another example, the raw sensor signal may be expected to exhibit a particular or predetermined profile or characteristic over a particular or predetermined period of time and the test condition may be met when the raw sensor signal is determined to match the expected profile or characteristic.

In some examples, the sensor electronics 1202 evaluates the test condition at least in part by examining an output of the deployment sensor 1210, for example to determine if the wireless signal 1214 received by the deployment sensor 1210 has changed to the deployed state. In some examples, testing for the deploy condition comprises a combination of examining the raw sensor signal and examining the output of the deployment sensor 1210. For example, the sensor electronics 1202 may find that the test condition is met if the raw sensor signal is greater than the deploy test threshold and the wireless signal 1214 has changed state. In another example, the sensor electronics 1202 may find that the test condition is met if the raw sensor signal is greater than the deploy test threshold or the wireless signal 1214 has changed state. If the test condition is not met at operation 1302, the sensor electronics 1202 may maintain the analyte sensor system 1200 in the sleep mode and return to operation 1302. For example, the sensor electronics 1202 may be configured to execute the operation 1302 periodically (e.g., every 5 minutes, every 30 seconds).

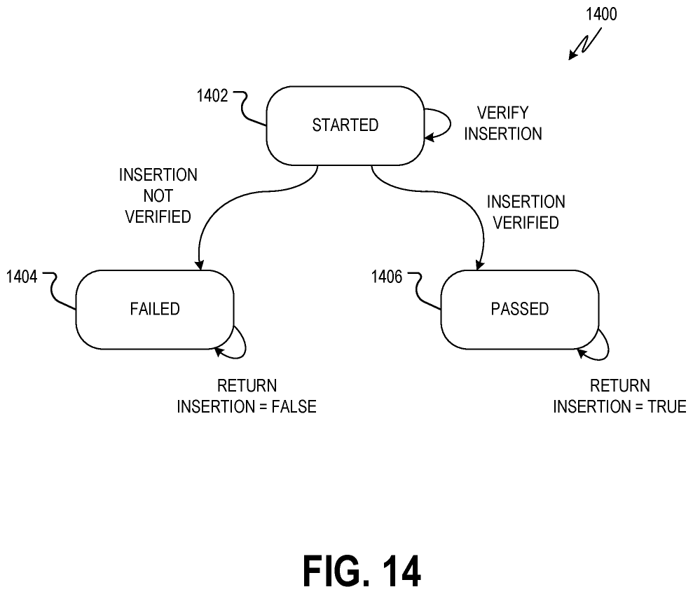

If the test condition is met at operation 1302, the sensor electronics 1202 determines at operation 1304 whether the analyte sensor system 1200 is ready for a sensor session. This may include examining one or more factors tending to indicate whether the analyte sensor 1206 has been inserted and is ready for use. For example, the sensor electronics 1202 may execute an insertion test to determine whether the raw sensor signal provided by the analyte sensor 1206 is consistent with the analyte sensor 1206 being positioned in vivo after insertion. For example, if the raw sensor signal remains above the deploy test threshold for a threshold time period, it may indicate that insertion of the analyte sensor 1206 has taken place. On the other hand, if the raw sensor signal drops below the deploy test threshold, it may indicate that the test condition was not prompted by a sensor insertion. One example insertion test is described herein with respect to FIG. 14.

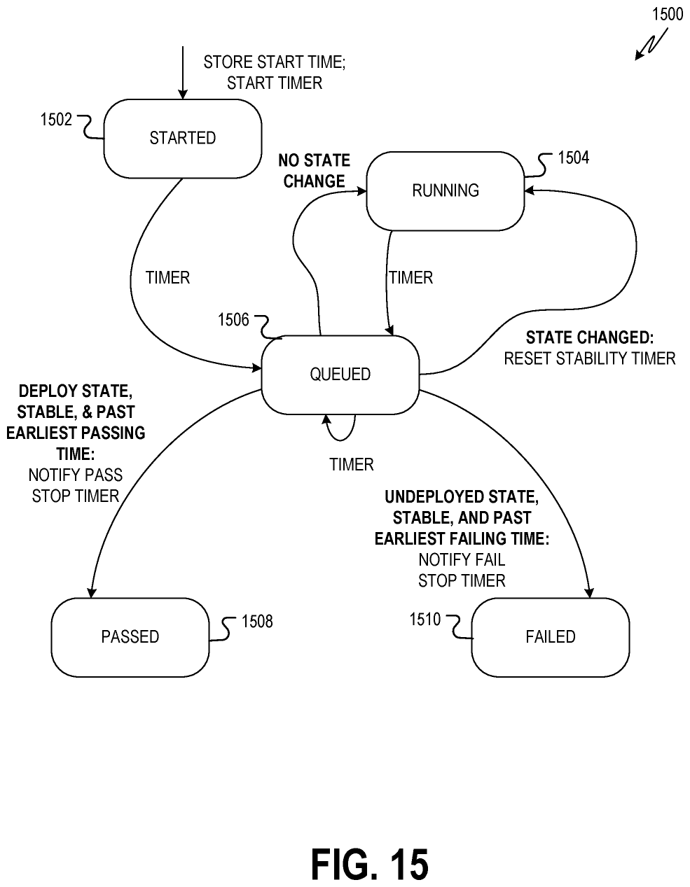

Also, in some examples, the sensor electronics 1202 executes a deploy test in addition to or instead of the insertion test. The deploy test may be based on the wireless signal 1214. For example, as described herein, when an applicator is used to insert the analyte sensor 1206 and/or is removed from the remainder of the analyte sensor system 1200, it may tend to change the state of the wireless signal 1214 from the undeployed state to the deployed state. The deploy test may include determining whether the wireless signal 1214 remains in one state for a stability threshold time period. If the wireless signal 1214 does remain in one state for the stability threshold time period, the sensor electronics 1202 may take a responsive action. For example, if the state of the wireless signal 1214 remains in the deployed state for a stability threshold time period, it may indicate that insertion of the analyte sensor 1206 has occurred and that the analyte sensor system 1200 is ready to transition to an active mode and begin a sensor session. The responsive action may include transitioning to an active mode by initiating a sensor session as described herein. On the other hand, if the wireless signal 1214 returns to the initial undeployed state for the stability threshold time period, it may indicate that the test condition was not prompted by a sensor insertion. The responsive action may include returning to a sleep mode. In some examples, if the wireless signal 1214 oscillates and fails to settle into any state for the stability threshold time period, it may indicate that the test condition was not prompted by a sensor insertion. The responsive action may include returning the analyte sensor system 1200 to the sleep mode. An example arrangement for implementing a deploy test is provided herein with respect to FIG. 15.

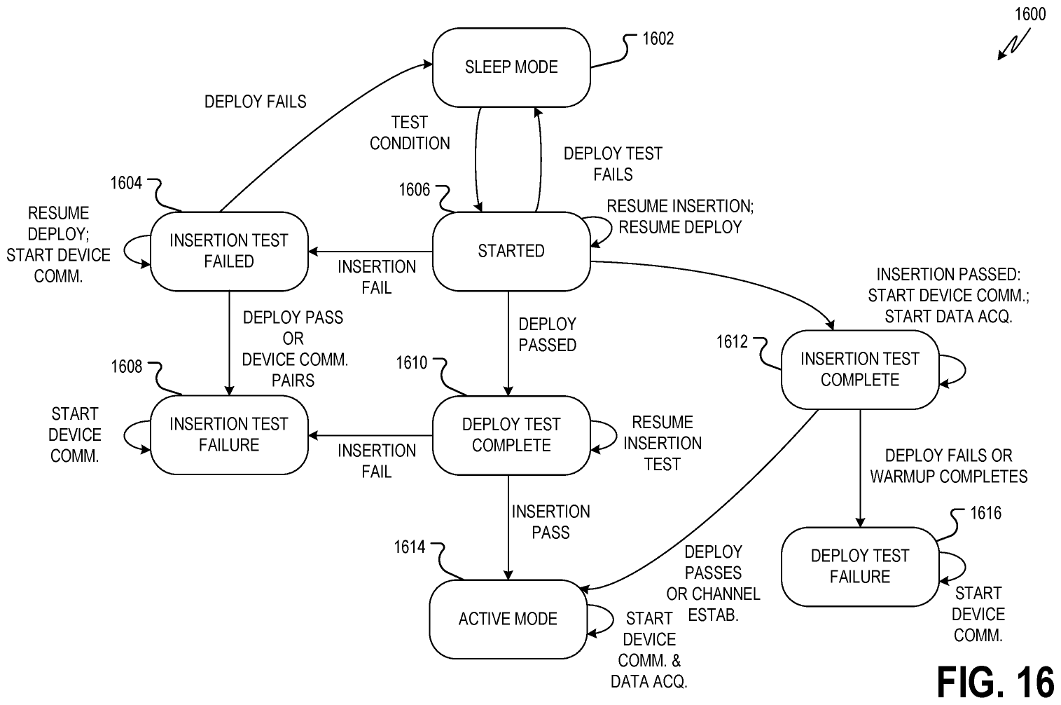

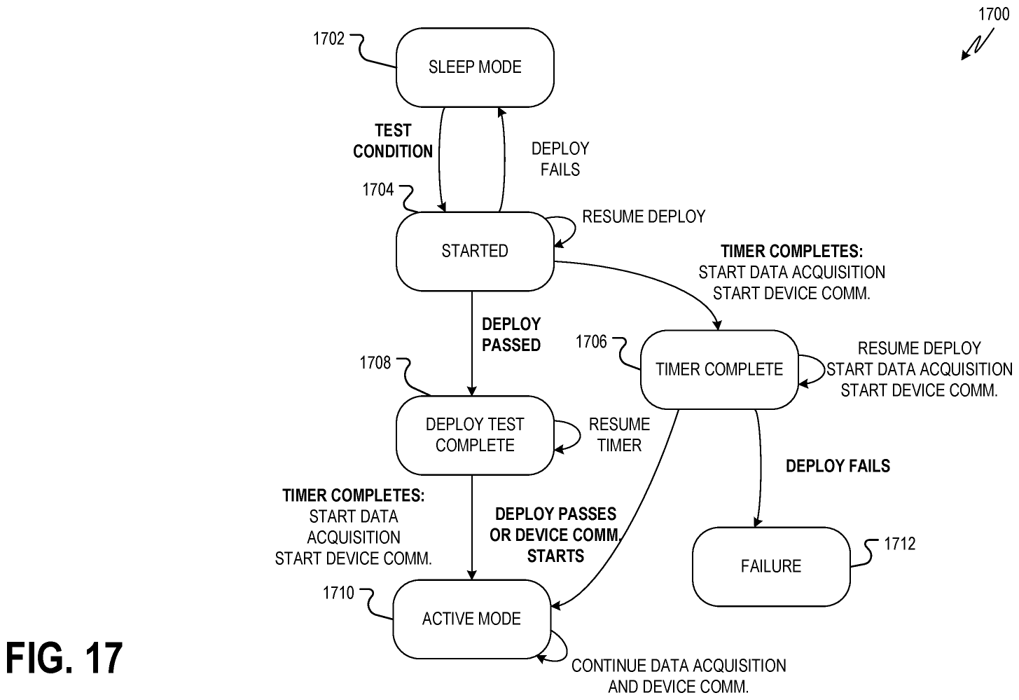

The sensor electronics 1202 may determine whether the analyte sensor system 1200 is ready for the active mode based on the insertion test, the deploy test or both. An example arrangement for determining whether the analyte sensor system 1200 is ready for the active mode using the insertion test and the deploy test is provided herein with respect to FIG. 16. An example arrangement for determining whether the analyte sensor system 1200 is ready for the active mode using the deploy test without the insertion test is provided herein with respect to FIG. 17.

If the deployment is not verified at operation 1304, the sensor electronics 1202 may return and/or maintain the analyte sensor system 1200 in the sleep mode at operation 1306 and then return to periodically test for the test condition at operation 1302. If the deployment is verified at operation 1304, the sensor electronics may transition to the analyte sensor system 1200 to an active mode at operation 1308, for example, by beginning a sensor session. Beginning the sensor session may include, for example, applying a bias condition to the analyte sensor 1206, initiating a warm-up or break-in period for the analyte sensor system 1200, and/or beginning to determine an estimated glucose value from the received raw sensor signal.

FIG. 14 is a state diagram showing one example of a workflow 1400 that may be executed by the sensor electronics 1202 of the analyte sensor system 1200 of FIG. 12 to perform an insertion test. This may include determining whether the raw sensor signal generated by the analyte sensor 1206 indicates that the analyte sensor 1206 has been inserted under a host's skin. As described herein, electrochemical activity at the analyte sensor 1206 may begin and/or increase when the analyte sensor 1206 is inserted in vivo. Accordingly, the raw sensor signal may also increase after the analyte sensor 1206 is inserted in vivo below the host's skin. In some examples, performing the insertion test includes detecting such an increase in the raw sensor signal.

The workflow 1400 may be executed when a test condition has occurred, for example, as described with respect to operation 1302. In some examples, the workflow 1400 shows one example way that the sensor electronics 1202 can execute all or part of the operation 1304 of the process flow 1300 of FIG. 13.

At a started state 1402, the sensor electronics 1202 tests the raw sensor signal generated by the analyte sensor 1206 to determine whether it indicates that the analyte sensor 1206 has been inserted in vivo. This can be performed, for example, by comparing the raw sensor signal to an insertion condition. The insertion condition may be, for example, a threshold signal level, whether the raw sensor signal maintains a threshold signal level for a threshold time, whether the raw sensor signal exhibits a threshold level of stability, etc. In some examples, the comparison of the raw sensor signal to the insertion condition may be performed after an insertion threshold time period has passed since the workflow 1400 was begun. This, for example, may allow the raw sensor signal from the analyte sensor 1206 to further increase as a result of being within the body for a period of time. Insertion may be verified if the raw sensor signal is above the threshold value after the insertion threshold time period.

If the insertion is not verified (e.g., the raw sensor signal does not meet the insertion condition), the sensor electronics 1202 proceed to a failed state 1404 and may return an indication that the insertion is not verified. If the insertion is verified (e.g., the raw sensor signal does meet the insertion condition), the sensor electronics 1202 may proceed to a passed state 1406 and may return an indication that the insertion is verified. The indication that the insertion is verified may be returned to another process and/or may be used to prompt the sensor electronics to transition the analyte sensor system 1200 to the active mode by starting a sensor session as described herein. In one embodiment, multiple insertion conditions may need to be met in order for the insertion to be verified.

FIG. 15 is a state diagram showing one example of a workflow 1500 that may be executed by the sensor electronics 1202 of the analyte sensor system 1200 of FIG. 12 to perform a deploy test to test for deployment of the analyte sensor 1206 using the wireless signal 1214 detected by the deployment sensor 1210. For example, the workflow 1500 shows one example way that the sensor electronics 1202 can execute all or part of the operation 1304 of the process flow 1300.

The workflow 1500 may detect when the wireless signal 1214 has settled into a stable state and return an indication of whether the stable state of the wireless signal 1214 is the deployed state or the undeployed state. If the wireless signal 1214 has settled into the deployed state indicative of sensor deployment, then the workflow 1500 may result in a determination that the deployment verification has passed. On the other hand, if the wireless signal 1214 has settled into the undeployed state indicative of no sensor deployment, then the workflow 1500 results in a determination that the deployment verification has failed.

At a started state 1502, the sensor electronics 1202 stores a start time and starts a stability timer. If it is not reset, the stability timer may run for a stability threshold time. For example, if the state of the wireless signal 1214 remains constant for the duration of the stability threshold time, it may indicate that the wireless signal 1214 is stable.

From the started state 1502, the sensor electronics 1202 may transition to a queued state 1506. At the queued state, the sensor electronics 1202 may continue to execute the stability timer. If in the queued state 1506, there is no change to the state of the wireless signal 1214, the sensor electronics 1202 may continue executing the stability timer in the running state 1504. If the state of the wireless signal 1214 does change, the sensor electronics 1202 resets the stability timer and runs the timer in the running state 1504 after the reset.

Consider an example in which the wireless signal 1214 is generated by a transmitter on the applicator, such as the transmitter 985 and/or the transmitter 1112. The execution of the workflow 1500 may have been prompted by the detection of a test condition such as an initial change in the state of the wireless signal 1214 to the deployed state and/or a change in the raw sensor signal. If the test condition was the result of an actual insertion of the analyte sensor 1206, then the transmitter may have changed the state of the wireless signal 1214 to the deployed state and/or the applicator (including the transmitter) may be physically removed from the vicinity of the analyte sensor system 1200, thereby affecting a durable change in the state of the wireless signal 1214 to the deployed state. In this case, the state of the wireless signal 1214 may remain stable in the deployed state. On the other hand, if the test condition was caused by a transitory and/or outside condition, the transmitter of the applicator may not have modified the state of the wireless signal 1214 to the deployed state, meaning that the wireless signal 1214 may oscillate and/or settle back to the undeployed state.

If the state of the wireless signal 1214 remains stable for the duration of the stability time period (e.g., if the stability timer expires without a change in the state of the wireless signal 1214), the sensor electronics 1202 may determine whether to proceed to a passed state 1508 and/or a failed state 1510, for example, based on which state the wireless signal 1214 has settled into and, in some examples, whether sufficient time has passed since the workflow 1500 was initiated. For example, if the wireless signal 1214 is stable in the deployed state, the sensor electronics 1202 may determine whether more than a passing time period has passed since the stability timer was initially started at the started state 1502. If more than the passing time period has passed, the sensor electronics 1202 may return a verification of sensor deployment, stop the timer, and proceed to the passed state 1508.

In some examples, the passing time period is less than or equal to the stability time period, meaning that any time the wireless signal 1214 is stable at the deployed state, the workflow 1500 may return a verification of insertion. In various other examples, however different passing time periods may be used, including passing time periods greater than the stability time period. For example, if the passing time period has not yet, passed, the sensor electronics 1202 may remain in the queued state 1506 until the passing time period has passed and/or the state of the wireless signal 1214 changes prompting the sensor electronics 1202 to reset the stability timer.

If the state of the wireless signal 1214 has remained stable for the duration of the stability time period in the undeployed time period, the sensor electronics 1202 may determine whether a failing time period has passed since the stability timer was initial started at the started state 1502. In some examples, the failing time period may be larger than the stability time period. If the failing time period has not passed, the sensor electronics 1202 may return to the queued state 1506 until the failing time period passes and/or the wireless signal 1214 changes state, prompting the sensor electronics 1202 to reset the stability timer. If the failing time period has passed, the sensor electronics may proceed to the failed state 1510. In the failed state 1510, the sensor electronics 1202 may return a failure of the insertion verification and stop the timer.

FIG. 16 is a state diagram showing an example workflow 1600 that may be executed by the sensor electronics 1202 to transition the analyte sensor system 1200 from a sleep mode to an active mode. In the example of FIG. 16, the sensor electronics 1202 verifies the deployment of the analyte sensor 1206 using an insertion test based on the raw sensor signal, for example, as described with respect to FIG. 14, and a deploy test based on the wireless signal 1214, for example, as described with respect to FIG. 15.

The sensor electronics 1202 may initially maintain the analyte sensor system 1200 in a sleep mode state 1602. If a test condition is met, the sensor electronics 1202 transitions to the started state 1606, where the insertion test and the deploy tests are run. In some examples, the insertion test and deploy test are executed concurrently. The test condition may be, for example, as described herein with respect to operation 1302 of the process flow 1300. If the deploy test fails while in the started state 1606, the sensor electronics 1202 may transition back to the sleep mode state 1602.

If the insertion test fails while in the started state 1606, the sensor electronics 1202 may transition to an insertion test failed state 1604. At the insertion test failed state 1604, the sensor electronics 1202 may continue to execute the deploy test and may also initiate a communication connection (e.g., a short-range wireless medium communication connection) with an external device, such the user computing device 132, peripheral medical device 122, or other external device. For example, the analyte sensor system 1200 may provide result data (e.g., estimated analyte values) to the external device for display to the host or other user. In examples where the short-range wireless medium is a Bluetooth® or Bluetooth LE® medium, initiating a communication connection with an external device may include pairing the sensor electronics to the external device.

From the insertion test failed state 1604, if the deploy test fails, the sensor electronics 1202 may transition back to the sleep mode state 1602. If the deploy test passes from the insertion test failed state 1604 and/or the sensor electronics 1202 successfully initiates a communication connection to an external device, the sensor electronics 1202 transition to an insertion test failure state 1608. In some examples, the sensor electronics 1202 may maintain or continue to attempt a communication channel with an external device while in the insertion test failure state. For example, if a communication channel is successfully established, the sensor electronics 1202 may utilize the communications channel to report failure of the insertion test to the external device.

From the started state 1606, if the insertion test passes, the sensor electronics may transition to an insertion test complete state 1612. At the insertion test complete state 1612, the sensor electronics 1202 may begin to initiate a communication connection (e.g., a short-range wireless medium communication channel) with an external device and may begin data acquisition from the analyte sensor 1206. This may include, for example, logging values of the raw sensor signal and/or processing the raw sensor signal to generate corresponding estimated analyte values. If, from the insertion test complete state 1612, the deploy test passes and/or the communication connection is established with the external device, the sensor electronics 1202 transitions to the active mode state 1614. At the active mode state 1614, the sensor electronics 1202 may operate the analyte sensor 1206 and the remainder of the analyte sensor system 1200 to capture and report estimated analyte values, as described herein.

If the deploy test fails from the insertion test complete state 1612, the sensor electronics 1202 may transition to a deploy test failure state 1616. At the deploy test failure state 1616, in some examples, the sensor electronics 1202 may continue to attempt a communication connection with an external device. For example, if a communication connection with the external device is successfully established, the sensor electronics 1202 may report the failure of the deploy test.

If from the started state 1606, the deploy test passes, the sensor electronics 1202 may transition to a deploy test complete 1610. At the deploy test complete state 1610, the sensor electronics 1202 may resume and/or otherwise continue the insertion test. If the insertion test fails from the deploy test complete state 1610, the sensor electronics may transition to the insertion test failure state 1608. If the insertion test passes from the deploy test complete state, the sensor electronics 1202 may transition to the active mode state 1614.

FIG. 17 is state diagram showing another example workflow 1700 that may be executed by the sensor electronics 1202 to transition the analyte sensor system 1200 from a sleep mode to an active mode. In the example of FIG. 17, the sensor electronics 1202 verifies the deployment of the analyte sensor 1206 using a deploy test based on the wireless signal 1214, for example, as described with respect to FIG. 15 and a startup timer. For example, in the workflow 1700, it may be assumed that if more than a startup time period has passed since the detection of the test condition, the analyte sensor 1206 is inserted and the analyte sensor system 1200 is ready for a sensor session.

The sensor electronics 1202 may initially maintain the analyte sensor system 1200 in a sleep mode state 1702. If a test condition is met, the sensor electronics 1202 transitions to the started state 1704, where the deploy test are run. The test condition may be, for example, as described herein with respect to operation 1302 of the process flow 1300. In some examples upon detecting the test condition, sensor electronics begin a startup timer. If the deploy test fails while in the started state 1704, the sensor electronics 1202 may transition back to the sleep mode state 1702.

If the startup timer completes from the started state 1704 (e.g., the startup time period has passed before the deploy test has either passed or failed), the sensor electronics 1202 may transition to a timer complete state 1706. At the timer complete state 1706, the sensor electronics 1202 may continue the deploy test and may also attempt a communication connection (e.g., a short-range wireless medium communication channel) with an external device and may begin data acquisition from the analyte sensor 1206. This may include, for example, logging values of the raw sensor signal and/or processing the raw sensor signal to generate corresponding estimated analyte values.

If, from the timer complete state 1706, the deploy test passes and/or the communication connection is established with the external device, the sensor electronics 1202 transitions to the active mode state 1710. At the active mode state 1710, the sensor electronics 1202 may operate the analyte sensor 1206 and the remainder of the analyte sensor system 1200 to capture and report estimated analyte values, as described herein. If the deploy test fails from the timer complete state 1706, the sensor electronics 1202 may transition to a failure state 1712. At the failure state 1712, the sensor electronics may continue to attempt a communication connection with an external device, for example, to report failure of the deploy test.

If from the started state 1704, the deploy test passes, the sensor electronics 1202 may transition to a deploy test complete state 1708. At the deploy test complete state 1708, the sensor electronics 1202 may resume and/or otherwise continue the startup timer. When the startup timer is complete (e.g., the startup time period has passed), the sensor electronics 1202 may transition to the active mode state 1710.

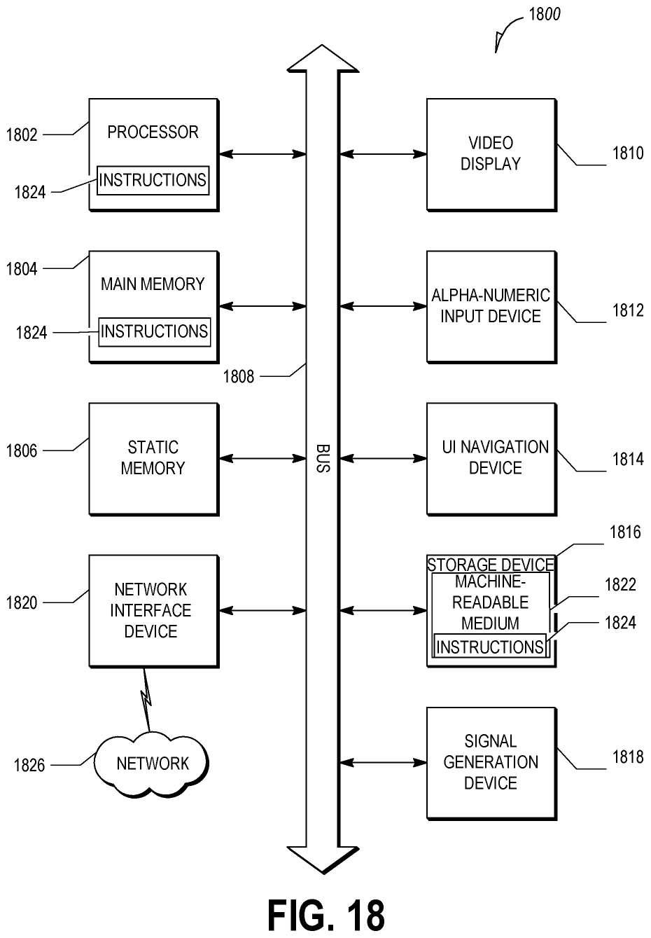

FIG. 18 is a block diagram illustrating a computing device hardware architecture 1800, within which a set or sequence of instructions can be executed to cause a machine to perform examples of any one of the methodologies discussed herein. The hardware architecture 1800 can describe various computing devices including, for example, the sensor electronics 106, the peripheral medical device 122, the smart device 112, the tablet 114, etc.

The architecture 1800 may operate as a standalone device or may be connected (e.g., networked) to other machines. In a networked deployment, the architecture 1800 may operate in the capacity of either a server or a client machine in server-client network environments, or it may act as a peer machine in peer-to-peer (or distributed) network environments. The architecture 1800 can be implemented in a personal computer (PC), a tablet PC, a hybrid tablet, a set-top box (STB), a personal digital assistant (PDA), a mobile telephone, a web appliance, a network router, a network switch, a network bridge, or any machine capable of executing instructions (sequential or otherwise) that specify operations to be taken by that machine.

The example architecture 1800 includes a processor unit 1802 comprising at least one processor (e.g., a central processing unit (CPU), a graphics processing unit (GPU), or both, processor cores, compute nodes). The architecture 1800 may further comprise a main memory 1804 and a static memory 1806, which communicate with each other via a link 1808 (e.g., bus). The architecture 1800 can further include a video display unit 1810, an input device 1812 (e.g., a keyboard), and a UI navigation device 1814 (e.g., a mouse). In some examples, the video display unit 1810, input device 1812, and UI navigation device 1814 are incorporated into a touchscreen display. The architecture 1800 may additionally include a storage device 1816 (e.g., a drive unit), a signal generation device 1818 (e.g., a speaker), a network interface device 1820, and one or more sensors (not shown), such as a Global Positioning System (GPS) sensor, compass, accelerometer, or another sensor.

In some examples, the processor unit 1802 or another suitable hardware component may support a hardware interrupt. In response to a hardware interrupt, the processor unit 1802 may pause its processing and execute an ISR, for example, as described herein.

The storage device 1816 includes a machine-readable medium 1822 on which is stored one or more sets of data structures and instructions 1824 (e.g., software) embodying or used by any one or more of the methodologies or functions described herein. The instructions 1824 can also reside, completely or at least partially, within the main memory 1804, within the static memory 1806, and/or within the processor unit 1802 during execution thereof by the architecture 1800, with the main memory 1804, the static memory 1806, and the processor unit 1802 also constituting machine-readable media.

### Executable Instructions and Machine-Storage Medium

The various memories (i.e., 1804, 1806, and/or memory of the processor unit(s) 1802) and/or storage device 1816 may store one or more sets of instructions and data structures (e.g., instructions) 1824 embodying or used by any one or more of the methodologies or functions described herein. These instructions, when executed by processor unit(s) 1802 cause various operations to implement the disclosed examples.

As used herein, the terms “machine-storage medium,” “device-storage medium,” “computer-storage medium” (referred to collectively as “machine-storage medium 1822”) mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 1822 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage media, computer-storage media, and device-storage media 1822 specifically exclude carrier waves, modulated data signals, and other such media, at least some of which are covered under the term “signal medium” discussed below.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer-Readable Medium

The terms “machine-readable medium,” “computer-readable medium” and “device-readable medium” mean the same thing and may be used interchangeably in this disclosure. The terms are defined to include both machine-storage media and signal media. Thus, the terms include both storage devices/media and carrier waves/modulated data signals.

The instructions 1824 can further be transmitted or received over a communications network 1826 using a transmission medium via the network interface device 1820 using any one of a number of well-known transfer protocols (e.g., HTTP). Examples of communication networks include a LAN, a WAN, the Internet, mobile telephone networks, plain old telephone service (POTS) networks, and wireless data networks (e.g., Wi-Fi, 3G, 4G LTE/LTE-A, 5G or WiMAX networks). The term “transmission medium” shall be taken to include any intangible medium that is capable of storing, encoding, or carrying instructions for execution by the machine, and includes digital or analog communications signals or other intangible media to facilitate communication of such software.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Various components are described in the present disclosure as being configured in a particular way. A component may be configured in any suitable manner. For example, a component that is or that includes a computing device may be configured with suitable software instructions that program the computing device. A component may also be configured by virtue of its hardware arrangement or in any other suitable manner.

The above description is intended to be illustrative, and not restrictive. For example, the above-described examples (or one or more aspects thereof) can be used in combination with others. Other examples can be used, such as by one of ordinary skill in the art upon reviewing the above description. The Abstract is to allow the reader to quickly ascertain the nature of the technical disclosure, for example, to comply with 37 C.F.R. § 1.72(b) in the United States of America. It is submitted with the understanding that it will not be used to interpret or limit the scope or meaning of the claims.

Also, in the above Detailed Description, various features can be grouped together to streamline the disclosure. However, the claims cannot set forth every feature disclosed herein, as examples can feature a subset of said features. Further, examples can include fewer features than those disclosed in a particular example. Thus, the following claims are hereby incorporated into the Detailed Description, with each claim standing on its own as a separate example. The scope of the examples disclosed herein is to be determined with reference to the appended claims, along with the full scope of equivalents to which such claims are entitled.

Each of these non-limiting examples in any portion of the above description may stand on its own or may be combined in various permutations or combinations with one or more of the other examples.

The above detailed description includes references to the accompanying drawings, which form a part of the detailed description. The drawings show, by way of illustration, specific embodiments in which the subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” “third,” etc., are used merely as labels, and are not intended to impose numerical requirements on their objects.

Geometric terms, such as “parallel”, “perpendicular”, “round”, or “square” are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the subject matter should be determined with reference to the claims, along with the full scope of equivalents to which such claims are entitled.

What is claimed is:

1. A sensor system for in vivo use, the sensor system comprising:
a sensor enclosure;
an analyte sensor extending from the sensor enclosure; and
sensor electronics positioned within the sensor enclosure, the sensor electronics being configured to perform operations comprising:
detecting that a wireless signal has changed from a first state to a second state, the wireless signal being provided through the sensor enclosure;
after detecting that the wireless signal has changed from the first state to the second state, monitoring whether the wireless signal remains in the second state for at least a stability threshold time period; and
executing an action in the sensor system, the action being selected based at least in part on whether the wireless signal remains in the second state for at least the stability threshold time period.

2. The sensor system of claim 1, further comprising an applicator for inserting the analyte sensor under a skin of a host, wherein the applicator comprises a transmitter, and wherein the transmitter generates the wireless signal.

3. The sensor system of claim 1, further comprising a wireless signal sensor, the wireless signal sensor comprising at least one of a magnetic signal sensor, an inductive signal sensor, or an optical signal sensor.

4. The sensor system of claim 1, the detecting that the wireless signal has changed from the first state to the second state comprising:
determining, at a first time that the wireless signal is in the first state at least in part by determining that the wireless signal is present; and
determining, at a second time, that the wireless signal is in the second state at least in part by determining that the wireless signal is absent, the second time being after the first time.

5. The sensor system of claim 1, the detecting that the wireless signal has changed from the first state to the second state comprising:
determining, at a first time, that the wireless signal is in the first state at least in part by determining that the wireless signal has a first magnetic polarity; and
determining, at a second time, that the wireless signal is in the second state at least in part by determining that the wireless signal has a second magnetic polarity different than the first magnetic polarity, the second time being after the first time.

6. The sensor system of claim 1, wherein determining whether the wireless signal remains in the second state for at least the stability threshold time period comprises determining that the wireless signal does remain in the second state for at least the stability threshold time period, and wherein the action comprises determining an estimated analyte value for a host using a sensor output generated by the analyte sensor.

7. The sensor system of claim 1, the operations further comprising determining that the sensor electronics have established a communication connection with an external device before the wireless signal has remained in the second state for at least the stability threshold time period, wherein the action comprises determining an estimated analyte value for a host using a sensor output generated by the analyte sensor.

8. The sensor system of claim 1, the operations further comprising after detecting that the wireless signal has changed from the first state to the second state, determining that the wireless signal has remained in the first state for more than the stability threshold time period, the action comprising configuring the sensor system to a sleep mode.

9. The sensor system of claim 1, the operations further comprising determining that a startup time period has passed since the detecting that the wireless signal has changed from the first state to the second state.

10. The sensor system of claim 1, the operations further comprising determining that more than a failure threshold time period has passed since the detecting that the wireless signal has changed from the first state to the second state, the failure threshold time period being greater than the stability threshold time period.

11. The sensor system of claim 1, the operations further comprising:
determining that a startup threshold time period has passed since the detecting that the wireless signal has changed from the first state to the second state; and
determining that the wireless signal has remained in the first state for more than the stability threshold time period, the action comprising configuring the sensor system to a failure state.

12. The sensor system of claim 1, the operations further comprising:
determining that a startup threshold time period has passed since the detecting that the wireless signal has changed from the first state to the second state and that the wireless signal has not remained in either the first state or in the second state for more than the stability threshold time period; and
initiating a communication connection with an external device.

13. The sensor system of claim 12, the operations further comprising determining that the sensor electronics have established the communication connection with the external device, wherein the action comprises determining an estimated analyte value for a host using a sensor output generated by the analyte sensor.

14. The sensor system of claim 1, the operations further comprising determining that a sensor output generated by the analyte sensor meets a first insertion condition.

15. The sensor system of claim 14, the operations further comprising:
after determining that the sensor output meets the first insertion condition, initiating a communication connection with an external device; and
determining that the sensor electronics have established the communication connection with the external device, wherein the action comprises determining an estimated analyte value for a host using a sensor output generated by the analyte sensor.

16. A method for operating an analyte sensor system comprising an in vivo analyte sensor, the method comprising:
detecting, by sensor electronics of the analyte sensor system, that a wireless signal has changed from a first state to a second state, the wireless signal being provided to the sensor electronics through a sensor enclosure of the analyte sensor system;
after detecting that the wireless signal has changed from the first state to the second state, monitoring, by the sensor electronics, whether the wireless signal remains in the second state for at least a stability threshold time period; and
executing, by the sensor electronics, an action in the analyte sensor system, the action being selected based at least in part on whether the wireless signal remains in the second state for at least the stability threshold time period.

17. The method of claim 16, further comprising receiving the wireless signal, by the sensor electronics and from a transmitter, the transmitter positioned at an applicator for inserting an analyte sensor of the analyte sensor system under a skin of a host.

18. The method of claim 16, further comprising receiving the wireless signal using at least one of a magnetic signal sensor, an inductive signal sensor, or an optical signal sensor.

19. The method of claim 16, the detecting that the wireless signal has changed from the first state to the second state comprising:
determining, at a first time that the wireless signal is in the first state at least in part by determining that the wireless signal is present; and
determining, at a second time, that the wireless signal is in the second state at least in part by determining that the wireless signal is absent, the second time being after the first time.

20. A non-transitory machine readable medium comprising instructions thereon that, when executed by an analyte sensor system, cause the analyte sensor system to perform operations comprising:
detecting that a wireless signal has changed from a first state to a second state, the wireless signal being provided to sensor electronics of the analyte sensor system through a sensor enclosure of the analyte sensor system;
after detecting that the wireless signal has changed from the first state to the second state, monitoring whether the wireless signal remains in the second state for at least a stability threshold time period; and
executing an action in the analyte sensor system, the action being selected based at least in part on whether the wireless signal remains in the second state for at least the stability threshold time period.
